(19) United States
(12) Patent Application Publication
(10) Pub. No.: US 2024/0161904 A1
(43) Pub. Date: May 16, 2024

(54) METHOD AND APPARATUS FOR ONLINE NUTRITIONAL SUPPLEMENT PLATFORM

(71) Applicant: PillWork Inc., Seoul (KR)

(72) Inventors: Young Gae CHO, Seoul (KR); So Ra NAM, Seoul (KR)

(73) Assignee: PillWork Inc., Seoul (KR)

(21) Appl. No.: 18/103,403

(22) Filed: Jan. 30, 2023

(30) Foreign Application Priority Data
Nov. 11, 2022 (KR) 10-2022-0150430

(51) Int. Cl. G16H 20/60; G16H 10/20

(52) U.S. Cl. CPC G16H 20/60; G16H 10/20

(57) ABSTRACT

A method for an online prescription platform may include providing main information based on user information, processing health information based on the user information, analyzing nutritional supplement ranking information based on the user information, and providing recommendation information and column information by receiving an input signal from a user.

### CROSS-REFERENCE TO RELATED APPLICATIONS

Pursuant to 35 U.S.C. § 119(a), this application claims the benefit of earlier filing dates and right of priority to Korean Application No. 10-2022-0150430, filed on Nov. 11, 2022, the contents of which are hereby incorporated by reference herein in their entirety.

### BACKGROUND

### Field

Embodiments relate to a method and apparatus for an online platform.

### Discussion of the Related Art

There are no platforms through which specialized information on drugs such as nutritional supplements is provided and drugs such as nutritional supplements are purchased. The global market for products such as nutritional supplements, especially the US market, is growing. However, it is difficult for consumers to easily use information on products such as nutritional supplements as compared to the global market size. Information is distributed between individual consumers on a peer-to-peer basis using a traditional offline method. Consumers do not have reliable information as compared to the market size. Accordingly, consumers need to rely on online and/or offline advertising information. There is a need for an online nutritional supplement platform service that provides consumers with reliable information and allows consumers to purchase nutritional supplements in a reliable manner based on the information.

### SUMMARY

Embodiments provide a method and apparatus for a nutritional supplement platform that connects consumers and professionals. Embodiments provide a method and apparatus for a platform that provides reliable nutritional supplements and health-related information. However, embodiments are not limited to the above-described technical objects, and the scope of embodiments may be extended to other technical objects that can be inferred by those skilled in the art based on the entire description.

A method for an online prescription platform according to embodiments is a method for an online nutritional supplement platform according to embodiments, including providing main information based on user information, processing health information based on the user information, analyzing nutritional supplement ranking information based on the user information, and providing recommendation information and column information by receiving an input signal from a user.

The method and apparatus for an online prescription platform according to embodiments can provide a platform capable of accurately and easily providing information on nutritional supplements based on expert information and allowing users to wisely consume nutritional supplements.

The method and apparatus for an online prescription platform according to embodiments can provide healthcare services that provide accurate and extensive health information by connecting consumers and experts.

The method and apparatus for an online prescription platform according to embodiments can provide objective, professional, and reliable healthcare services based on smart artificial intelligence (AI).

### BRIEF DESCRIPTION OF THE DRAWINGS

The drawings are included to provide a further understanding of embodiments and illustrate the embodiments along with description relating to the embodiments. For a better understanding of various embodiments described below, reference should be made to the description of the following embodiments in conjunction with the following drawings, which include corresponding parts with like reference numerals throughout the drawings.

FIG. 1 shows a relationship of examples of user interface (UI) screens according to embodiments.

FIG. 2 shows the screen of the menu according to embodiments.

FIGS. 3, 4, and 5 show main screens according to embodiments.

FIGS. 6, 7, 8, and 9 show the survey/result according to embodiments.

FIGS. 10 and 11 show the ranking according to embodiments.

FIG. 12 shows a detailed search and result screen according to embodiments.

FIGS. 13, 14 and 15 show the product details according to embodiments.

FIG. 16 shows the writing review according to embodiments.

FIG. 17 shows best combinations and a detailed view screen according to embodiments.

FIG. 18 shows a pharmacist column list screen and a detailed view screen according to embodiments.

FIG. 19 shows the My Page screen according to embodiments.

FIG. 20 shows the challenge according to embodiments.

FIGS. 21 and 22 shows the review and the save product list according to embodiments.

FIG. 23 illustrates a method for an online prescription platform according to embodiments.

FIG. 24 illustrates a method for an online prescription platform according to embodiments.

FIG. 25 shows an apparatus for an online prescription platform according to embodiments.

FIGS. 26A and 26B show an apparatus for the online prescription platform according to embodiments.

FIG. 27 illustrates a method for the online prescription platform according to embodiments.

FIG. 28 illustrates a method of providing the online platform service according to embodiments.

### DETAILED DESCRIPTION

Preferred embodiments will be described in detail, examples of which are shown in the accompanying drawings. The detailed description below with reference to the accompanying drawings is intended to describe preferred embodiments rather than only showing embodiments that can be implemented. The detailed description below includes details to provide a thorough understanding of the embodiments. However, it will be apparent to those skilled in the art that the embodiments can be practiced without these details.

Most terms used in the embodiments are selected from general ones widely used in the field, but some terms are arbitrarily selected by the applicant and their meanings are described in detail in the following description as needed. Therefore, the embodiments should be understood based on the intended meanings of the terms rather than the simple names or meanings of the terms.

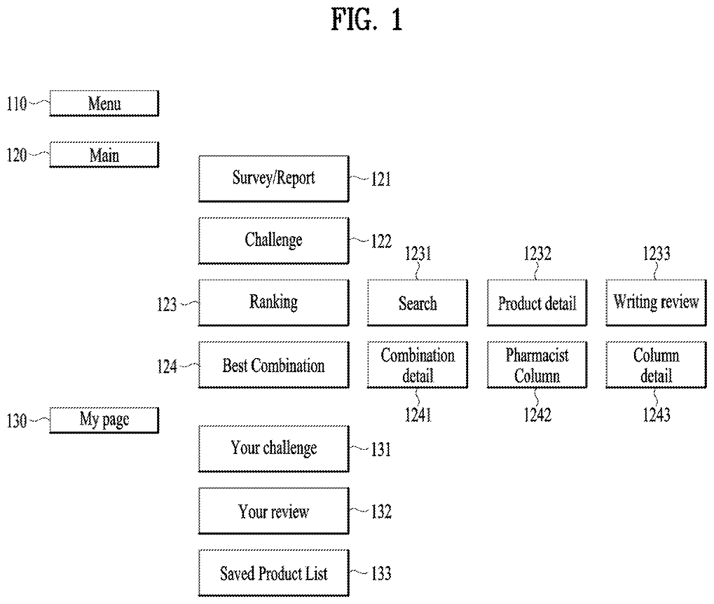

FIG. 1 shows a relationship of examples of user interface (UI) screens according to embodiments.

Referring to FIG. 1, examples of UI screens according to embodiments are screens that may be displayed by various display devices such as a terminal, a smartphone, and a mobile phone used by a client or a user.

In examples of UI screens according to embodiments, areas included in each screen may be configured by an application installed in an apparatus, and information may be displayed on the configured areas.

Referring to FIG. 1, UI screens according to embodiments may include a menu 110, a main screen 120, and My Page 130. In addition, the UI screens according to the embodiments may include a survey/result 121, challenge 122, ranking 123 and best combinations 124. In addition, the UI screens according to the embodiments may include search 1231, product details 1232, writing review 1233, combination details 1241, a pharmacist column list 1242, and column details 1243. Further, the UI screens according to the embodiments may include a challenge 131, a review 132, and a saved product list 133.

Referring to FIG. 1, transition may occur from the main screen 120 to the survey/result 121, challenge 122, ranking 123, and best combinations 124 and from My Page 130 to the challenge 131, review 132 and saved product list 133. In addition, transition may occur from the ranking 123 to the search 1231, product details 1232, and writing review 1233 and from the best combinations 124 to the combination details 1241, pharmacist column list 1242, and column details 1243.

The transition relationship and connection relationship between screens illustrated in FIG. 1 may be applied in various manners according to embodiments, and are not limited by the structure shown in FIG. 1.

FIG. 1 shows functions provided by a method and apparatus for an online prescription platform (providing online nutritional supplements) according to embodiments. In this specification, a method and apparatus for an online prescription platform according to embodiments may be referred to as a method/apparatus according to the embodiments.

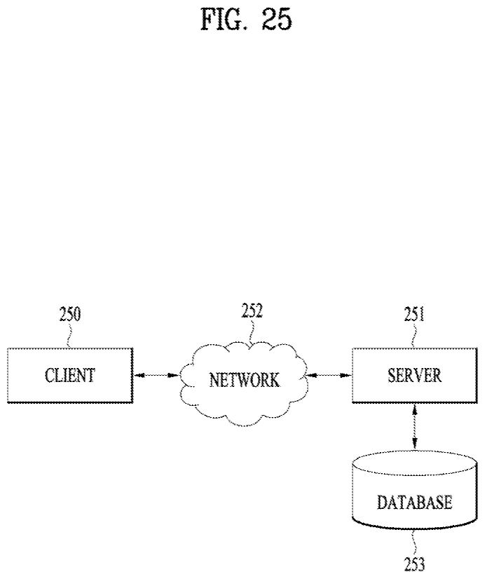

As shown in FIG. 1, the method/apparatus according to the embodiments may provide various functions. Information representing each function may be displayed as UI/UX (User Interface & User Experience). The method/apparatus according to the embodiments may operate in association with various display devices such as a terminal, a smartphone, and a mobile phone used by a client or a user. The apparatus according to the embodiments may correspond to a client 250, a server 251, a database 253, or the like shown in FIG. 25.

Each function in FIG. 1 and UI/UX information corresponding to each function may be processed and displayed by the apparatus according to the embodiments. Each function may be selectively displayed. Each function may be selected according to system settings. Each function may be selected based on an input signal of a user. The input signal of the user may be transmitted and received between the apparatus and the user by physical touch, an electrical signal, or a signal through a network. A term representing each function may be referred to by another term within the range having an equivalent meaning. Each function may have a hierarchical structure. Each function may have an initial value set by the system. Each function may be modified according to user's settings. A process of processing each function and a process of transition between functions may be performed according to user selection.

Hereinafter, each function will be described with reference to the drawings.

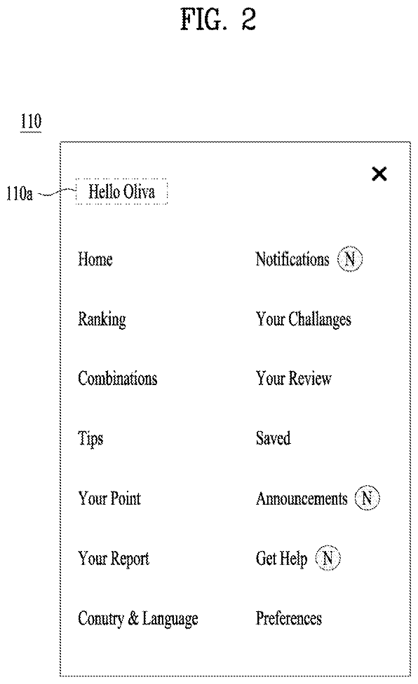

FIG. 2 shows the screen of the menu 110 according to embodiments.

Referring to FIG. 2, the menu 110 includes an area 110a representing a user ID (or nickname) at the top of the screen. In the area 110a, a greeting using a member ID may be represented in the case of a member who has signed up for and logged in to the service, and an icon for moving to a service sign-up screen may be located in the case of a user who has not signed up for the service.

As shown in FIG. 2, the menu 110 may display Home, Ranking, Combination, Tips, Your Points, Your Report, Notification, Your Challenges, Your Review, Saved, Announcements, Get Help, Country & Language, and Preferences icons. When a user clicks icons displayed on the menu 110, another page screen may be displayed by a click operation.

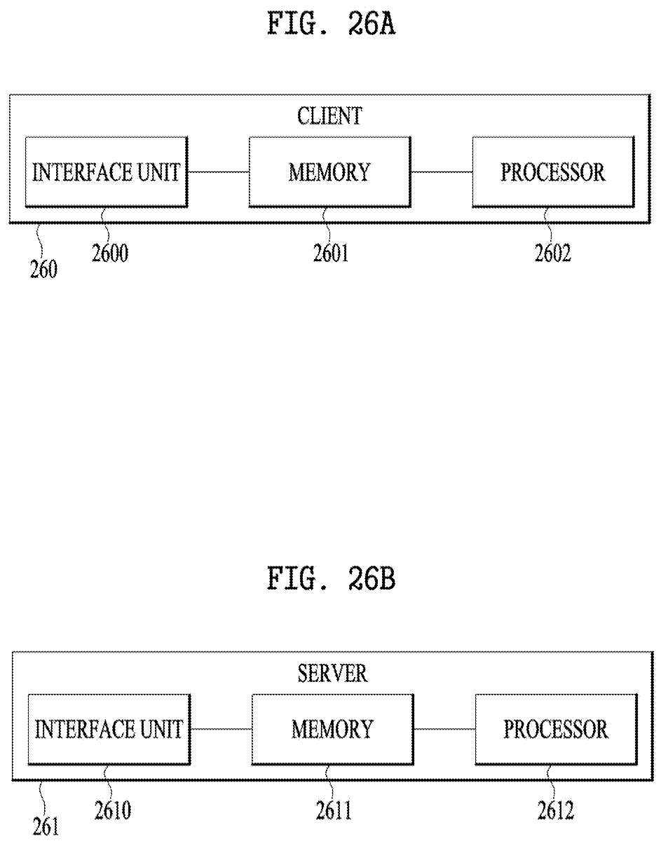

The icons displayed on the menu 110 in FIG. 2 are not limited to those shown in the figure, and the icons may be connected to the various screens described in FIG. 1 such that the user can move directly from the menu 110 to the various screens. Here, moving may mean that a device (e.g., a smartphone used by a user) detects an operation of the user, such as clicking or scrolling and displays another page screen or displays another area in response to the operation. A user terminal (or client, refer to FIGS. 25, 26A and 26B) may display information received from a server in a size, color, shape, font style, etc. at a position determined by an application program (e.g., application, etc.) like the UI screen according to embodiments.

The method/apparatus according to the embodiments may provide the menu 110 to a user. The menu 110 shown in FIG. 2 corresponds to the menu 110 shown in FIG. 1. The method/apparatus according to the embodiments may generate the menu 110 as shown in FIG. 2 for user convenience. In the method/apparatus according to the embodiments, data stored in the database 253 and the server 251 shown in FIG. 25 may be received through a network 252, and the client 250 may provide the menu 110 and information required for the menu 110. Each function of the menu shown in FIG. 2 may be selectively provided according to an input signal of the user and/or system settings. UI/UX information indicating each function may be displayed as shown in FIG. 2 in various orders, positions, sizes, and the like. The method/apparatus according to the embodiments may generate a new icon as shown in FIG. 2 when data is updated in relation to each function.

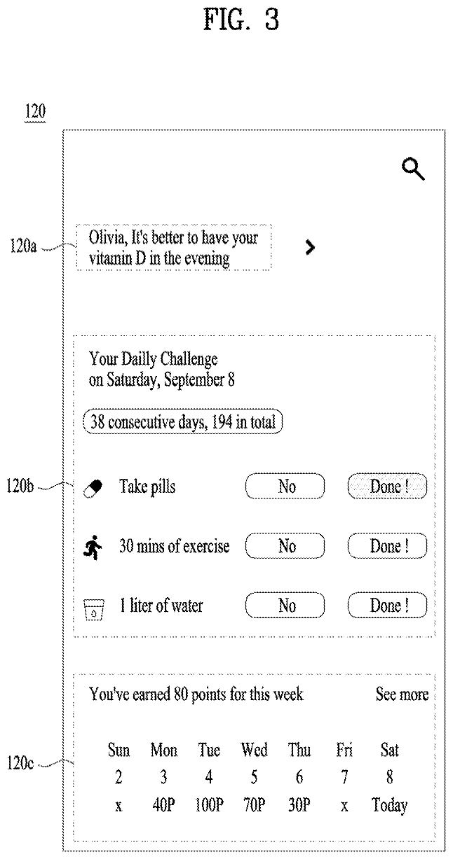

FIGS. 3, 4, and 5 show main screens 120 according to embodiments.

Referring to FIG. 3, the main screen 120 may include an area 120a representing the ingredients of a product and an intake time along with a user ID. The text displayed in the area 120a may indicate the type and ingredients of the product and the intake time based on user's survey results. For example, when the user has replied to a survey by inputting information representing that he/she is taking lutein, “{User nickname}, it is better to take your lutein before going to bed.” may be displayed. If there is no survey information, it is possible to display general text for health care for each of morning, lunch, and evening time periods. Here, the product may be a nutritional supplement product.

When the user clicks the area 120a, the user may move to the survey/result screen and reply to a survey or move to a screen displaying results of a survey that has already been performed. That is, the method/apparatus according to the embodiments may display a survey result screen linked to the area 102a.

The main screen 120 may include an area 120b representing information on user's challenges. The area 120b displays information on user's challenge performance based on the current date and may display how many days a challenge was completed consecutively and how many times the challenge was completed in total.

Types of challenge may include whether the user takes a nutritional supplement, whether the user exercises, whether the user drinks sufficient water, and the like. In addition, the area 120b displays an icon “No” and an icon “Done” indicating whether the user has completed execution of each challenge item such that the user can click the icons according to whether the user has performed challenges. The color of a clicked icon may be changed to indicate whether or not the icon is clicked, and information on whether the user has performed a challenge may be transmitted to and stored in the server or the database or converted into points through calculation.

In addition, the main screen 120 may include an area 120c displaying points acquired by the user based on a specific period (e.g., one week). The area 120c may display point information obtained by the user based on challenge execution information of the user on a day-to-day basis, and days on which the point information is displayed may be displayed on a weekly basis. The point information displayed in the area 120c may be calculated by the server based on the challenge performance information of the user.

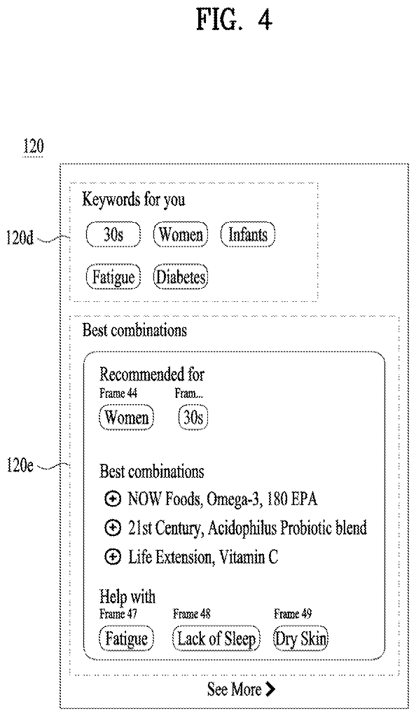

Referring to FIG. 4, the main screen 120 may include an area 120d displaying keywords related to the user and an area 120e displaying information on best combinations.

The area 120d displays a keyword representing the age of the user, a keyword representing the sex of the user, a keyword representing whether the user has a child, a keyword representing the age of the child, a keyword related to diseases, and the like based on survey results of the user. The user can click corresponding keywords and move to a screen displaying a collection of products (nutritional supplement products) related to the clicked keywords through clicking on the keywords. That is, the method/apparatus according to the embodiments may display a screen on which products (nutritional supplement products) related to clicked keywords are displayed.

The area 120e is an area representing information on combinations of products that are good to take together based on survey results of the user. The area 120e may display keywords (for example, “female” and “30s”) for a target for which combined products are recommended and keywords (for example, “fatigue,” “lack of sleep,” and “dry skin”) related to symptoms that can be improved by the combined products.

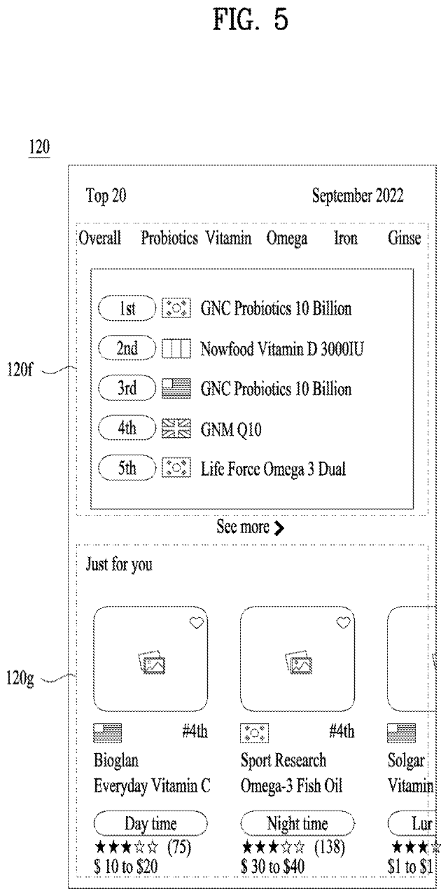

Referring to FIG. 5, the main screen 120 includes an area 120f displaying information on ranking of products and an area 120g displaying information on recommended products.

The area 120f may display the ranking, the countries of origin of products, and product names together and may indicate the ranking according to categories of nutritional components. For example, the ranking of products belonging to a vitamin category may be displayed. The area 120f includes an icon “see more”, and the user can move to the ranking screen 123 by clicking the icon “see more.” The method/apparatus according to the embodiments may display the ranking screen 123 when the user clicks the icon “see more” in the area 120f.

The 120g area may display images of recommended products, images of countries of origin, ranking information, names, major nutritional components, recommended intake times (for example, day, night, lunch, dinner, etc.), rating information, and price range information. Information on recommended products is arranged in the form of a card such that information on each product can be moved on the screen by a lateral sliding operation. The method/apparatus according to the embodiments may switch screens or display another page screen in response to a scrolling (or sliding) operation of the user.

The method/apparatus according to the embodiments may provide the function of the main screen 120, as shown in FIGS. 3, 4, and 5. The function of the main screen 120 of FIGS. 3, 4 and 5 corresponds to the function of the main screen 120 of FIG. 2.

Referring to FIG. 3, the method/apparatus according to the embodiments may generate the message 120a and provide the same to the user. For example, when the user's name stored in the database and/or the server is Olivia, the method/apparatus according to the embodiments may generate information such as “It's better to take your vitamin D in the evening” through a message UI/UX and display the message. The message 120a may be received from the database or the server, or generated by a client. The method/apparatus according to the embodiments may generate and receive the information 120b on user's challenges and provide the information 120b to the user. The information 120b on user's challenges may be received from the database or the server, or be generated by a client. The method/apparatus according to the embodiments may generate the information on user's challenges based on user information and/or expert information. Based on information on body information and/or health information of the user, nutritional supplement intake information, exercise information, lifestyle information, and the like necessary for the user may be generated and provided to check whether or not the user has performed challenges. The body information and/or the health information of the user may be stored in the database and may be received from the server. Information related to a reward may be provided to the user according to whether the user has accomplished a challenge. If the user does not achieve the challenge, related information may be provided such that the user can achieve the challenge. The method/apparatus according to the embodiments may receive data regarding user's challenges from the user and store the data in a client memory, the server, the database, and the like. The method/apparatus according to the embodiments may provide information on results of execution of user's challenges through the client using user information stored in the database and/or operation of the server. Challenge execution result information UI/UX may be displayed based on time, date, score, and the like.

The method/apparatus according to the embodiments may generate challenge items based on user information and display the generated challenge items in the area 120b of the main screen 120. The challenge items may include taking nutritional supplements, exercising for 30 minutes, and drinking 1L of water. In addition, the method/apparatus according to the embodiments may display icons indicating whether each challenge item has been performed along with each challenge item. The user may click an icon for each challenge item to input whether he/she has performed the same. Input information regarding whether the user has performed a challenge may be transmitted to and stored in the server or the database, or may be used to calculate reward points.

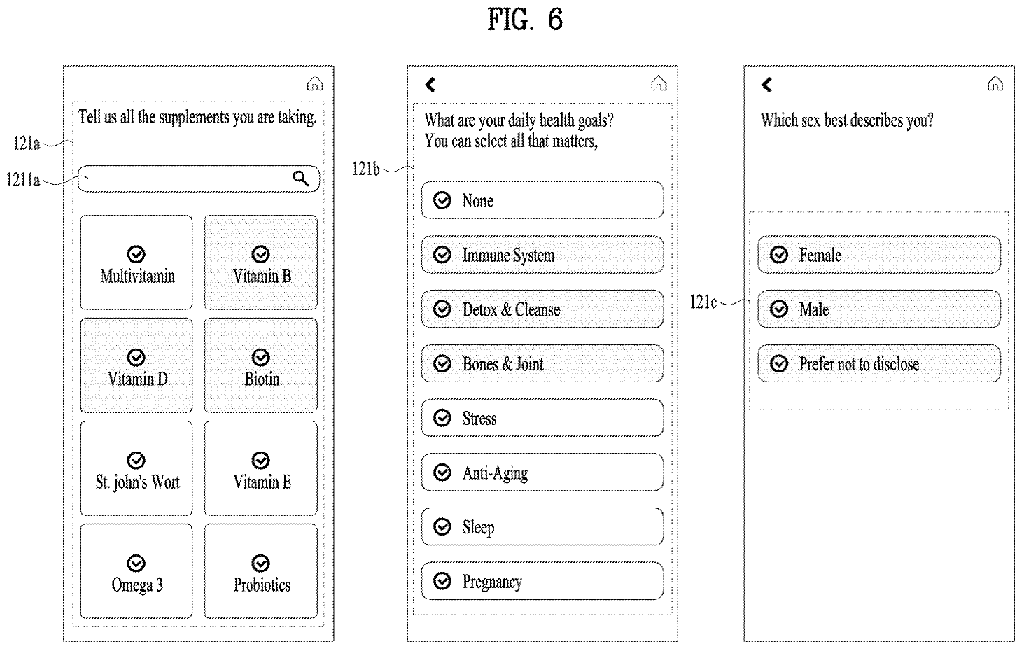

The user information used to generate the challenge items is based on details of surveys for the user (refer to FIGS. 6 to 8). Challenge items may be generated based on the user information in the client, the server, or the database.

Referring to FIG. 4, the method/apparatus according to the embodiments may store user information in the client, the server, the database, and the memory, optimize the stored user information, and display the user information to the user. The user information may be filtered based on recent activities of the user. The user information may be displayed based on keywords related to the user. The method/apparatus according to the embodiments may process and provide many users and big data of users based on keywords. Information related to the user may be converted into keywords based on frequency. The method/apparatus according to the embodiments may generate, store, and provide best combination information based on the user information. The best combination information may refer to data optimized for the user generated based on the health information and lifestyle information of the user. It is possible to provide the user with a combination of nutritional supplement information, exercise, and eating habit information most required for the user at the present time. The method/apparatus according to the embodiments may use the user information stored in the memory. In the method/apparatus according to the embodiments, the server may generate best health information based on data regarding a plurality of users stored in the database. The server may deliver information optimized for the user to the client. The method/apparatus according to the embodiments may generate objective and accurate best combination information using big data accumulated not only from an individual user but also from a plurality of users. In addition, the method/apparatus according to the embodiments may collect diagnostic information from experts with specialized health knowledge, such as doctors and pharmacists, optimize the collected diagnostic information, and provide the same to the user. Accordingly, the method/apparatus according to the embodiments can rapidly provide objective and accurate health information to the user. According to the embodiments, the best combination information may be generated based on the user's age, health condition, eating habits, sex, currently taken nutritional supplements, drug information, past nutritional supplements, and drug information. The best combination information may include comprehensive body information such as skin condition as well as nutritional supplement information, eating habits, and exercise information.

The method/apparatus according to the embodiments may generate nutritional supplement combination information based on the user information and display the generated nutritional supplement combination information on the are 120e of the main screen 120. The nutritional supplement combination information may list names of a plurality of nutritional supplements used in combination. In addition, the method/apparatus according to the embodiments may also display keywords for symptoms for which the displayed nutrient combination information can be helpful in the area 120e. The user may click the displayed nutritional supplement combination information to move to a detailed view page or a purchase page for nutritional supplements. That is, the method/apparatus according to the embodiments may display a detailed view screen or a purchase screen of related nutritional supplements in response to an operation of clicking the nutritional supplement combination information.

The user information used to generate the nutritional supplement combination information may be based on details of surveys for the user (refer to FIGS. 6 to 8) or information recommended by experts. Further, the nutritional supplement combination information may be information registered by an administrator.

The nutritional supplement combination information may include nutritional components that are beneficial when consumed together with nutritional components that the user is currently taking based on the details of surveys for the user. Alternatively, nutritional components that are unfavorable when consumed together with the nutritional components that the user is currently taking may be excluded. The nutritional supplement combination information may be generated in consideration of user's pregnancy status, lifestyle, age, ingredient content of nutritional supplements that the user is taking, and the like according to the details of surveys for the user.

Referring to FIG. 5, the method/apparatus according to the embodiments may provide overall nutritional supplement ranking information to the user based on usage data accumulated from users using the present platform service. Through platform data used by users of various nationalities, sexes, ages, and the like, it is possible to provide a nutritional supplement ranking as overall ranking and ranking by types. The user can view ranking information and use reliable platform information. The method/apparatus according to the embodiments may provide nutritional supplement recommendation information optimized for the current user. The method/apparatus according to the embodiments may provide health information and nutritional supplement recommendation information required for the current user for each nutritional supplement type. Here, it is possible to provide reliable health/nutrition information to the current user using data used by the current user, data used by users having attributes similar to the current user, and expert opinion data regarding the current user and users similar to the current user. The method/apparatus according to the embodiments may display the ranking of a product, information indicating where a nutritional supplement product is manufactured/sold, information on a nutritional supplement intake time, evaluation information of users who have purchased nutritional supplements, information on components of nutritional supplements, and the like. It is possible to generate recommendation information optimized for a user based on past, present, and future prediction data of the user, past, present, and future prediction data of a plurality of users, and expert opinion data.

FIGS. 6, 7, 8, and 9 show the survey/result 121 according to embodiments.

Referring to FIG. 6, the survey/result 121 includes an area 121a for selecting nutritional components to be consumed by the user.

The area 121a displays a plurality of icons indicating various nutritional components along with text for guiding a survey, and whether or not the corresponding icons are selected may be displayed by clicking by the user. The user may select a plurality of icons. In addition, the area 121a includes an input window 1211a through which text can be input, and the user can directly input keywords to the input window 1211a to search for nutritional ingredients to be consumed. The area 121a may include an icon for moving to the next step of the survey, and when the user clicks this icon, the screen can be switched to a screen representing the next survey.

An area 121b may display icons indicating diseases or conditions that the user wants to improve along with text for guiding the survey, and the user may select a plurality of icons. The selected icons are displayed such that the user can ascertain whether or not they are selected. The icons may include “none,” “immune system,”, “detox & cleanse,” “bones and joints,” “stress,” “anti-aging,” “sleep,” “pregnancy,” etc.

An area 121c displays icons indicating the user's sex along with text for guiding the survey. Selected icons are displayed such that the user can ascertain whether or not they are selected. The icons may include “female,” “male,” “prefer not to disclose,” etc.

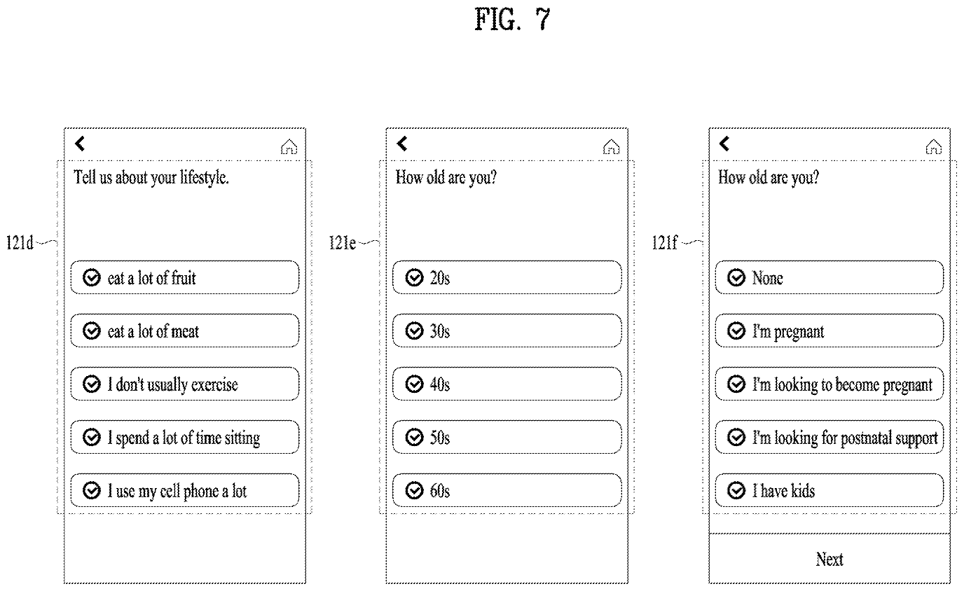

Referring to FIG. 7, the survey/result 121 may include an area 121d for surveying the user's living environment, an area 121e for surveying the age of the user, and an area 121f for surveying other user's conditions.

The areas are configured on different pages such that the user can move to the next page by clicking a survey completion icon. Alternatively, the areas may be included in one page such that the user can move to areas through scrolling.

The area 121d displays items for surveying the user's lifestyle. The items are items for checking the user's lifestyle, environment, style, and the like, such as “eat a lot of fruit,” “eat a lot of meat,” “I don't usually exercise,” and “I spend a lot of time sitting.” The user may select at least one corresponding item from the items.

The area 121e displays items for surveying the user's age group. The items may include “20s,” “30s,” “40s,” “50s,” “60s and older,” etc.

The area 121f displays items for surveying other characteristics related to the user. The items include items related to pregnancy and childbirth, such as “none,” “I'm pregnant,” and “I have kids.”

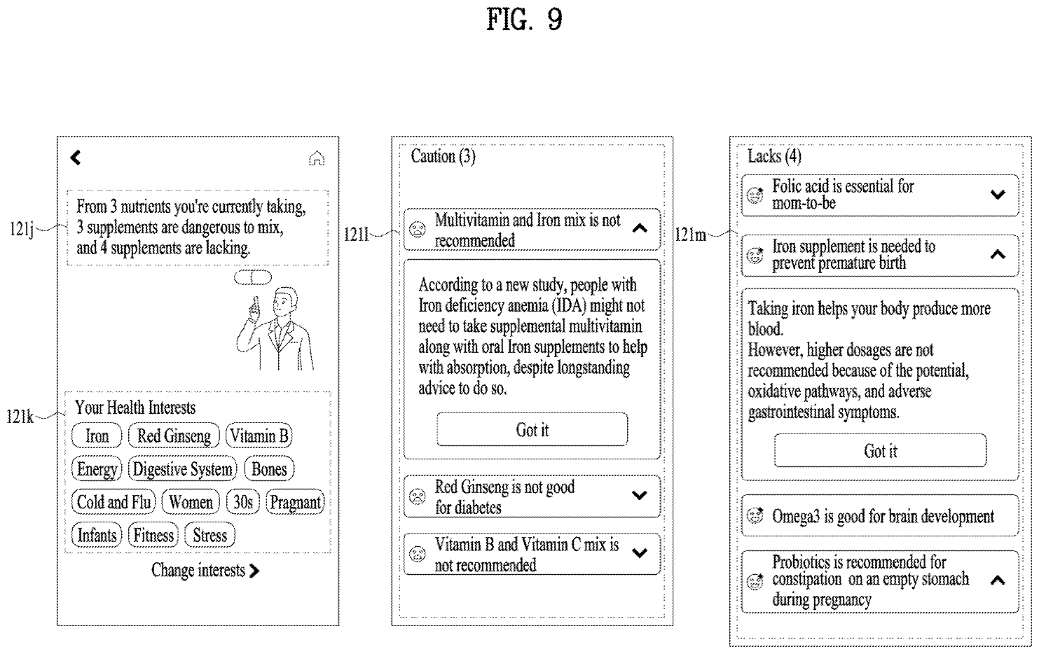

Referring to FIG. 9, the survey/result 121 may include an area 121j for summarizing survey results and displaying the same as text, an area 121k for keywords related to the user, an area 121l for cautions, and an area 121m displaying a lack of nutritional supplement.

The area 121j may display the types (number) of nutritional components that the user is taking, the number of components that are dangerous to mix, and the number of nutritional components that are lacking as text. For example, text of “Of the 3 nutrients you're currently taking, 3 supplements are dangerous to mix, and 4 supplements are lacking.” May be displayed.

The area 121k displays keywords related to the user or keywords in which the user is interested. Types of keywords may include nutritional components, diseases or conditions, symptoms, age group, sex, pregnancy status, and the like.

The area 121l displays cautions for the user. The area 121l displays a combination of nutritional supplements that are not recommended based on the surveys for the user. For example, a box including text such as “multivitamin and iron mix is not recommended,” “red ginseng is not good for diabetes,” and “vitamin B and vitamin C mix is not recommended.” may be displayed. The user can click an icon located next to each text to check the details. When the user clicks an icon, an additional box appears at the bottom of the corresponding text, and a detailed description is included in the box.

The area 121m displays details of nutritional components that the user is likely to lack. The area 121m displays nutritional components that the user is likely to lack as text based on the surveys for the user. For example, “Folic acid is essential for mom-to-be” and “Iron supplement is needed to prevent premature birth” may be displayed. Each text is located in a box, and boxes containing text are arranged in a vertical direction. The user can click an icon located next to text to check the details. When the user clicks an icon, an additional box appears at the bottom of the corresponding text, and a detailed description is included in the box.

Referring to FIG. 6, the method/apparatus according to the embodiments may receive information from the user through the client 250 (260) and/or an interface unit 2600 of the client. The client and/or the interface unit according to the embodiments may receive intake nutritional supplement information from the user through a UI/UX icon as shown in FIG. 6. As shown in FIG. 6, the UI/UX icon may be information indicating multivitamin, vitamin B, vitamin D, and the like. In addition to the UI/UX icon, the method/apparatus according to the embodiments may receive information directly input by the user, search the memory and/or the database for data corresponding to the input information of the user, and filter necessary data. The method/apparatus according to the embodiments may receive daily health goal information of the user from the user through the client and/or the interface unit of the client. User information may be received by generating a UI/UX icon indicating the daily health goal information of the user through the client and/or a processor according to the embodiments. The apparatus according to the embodiments may receive one or more pieces of user selection information. Similarly, the apparatus according to the embodiments may receive information on the user's sex. As user's personal information, sex, age, body, health information may be received.

Referring to FIG. 7, the method/apparatus according to the embodiments may generates UI/UX icons indicating user's lifestyle, age, life information, and the like through the client and/or the interface unit, and receive user information through the UI/UX icons. User information according to the embodiments illustrated in FIGS. 6 to 10 may be stored in the memory, the server, the database, and the like.

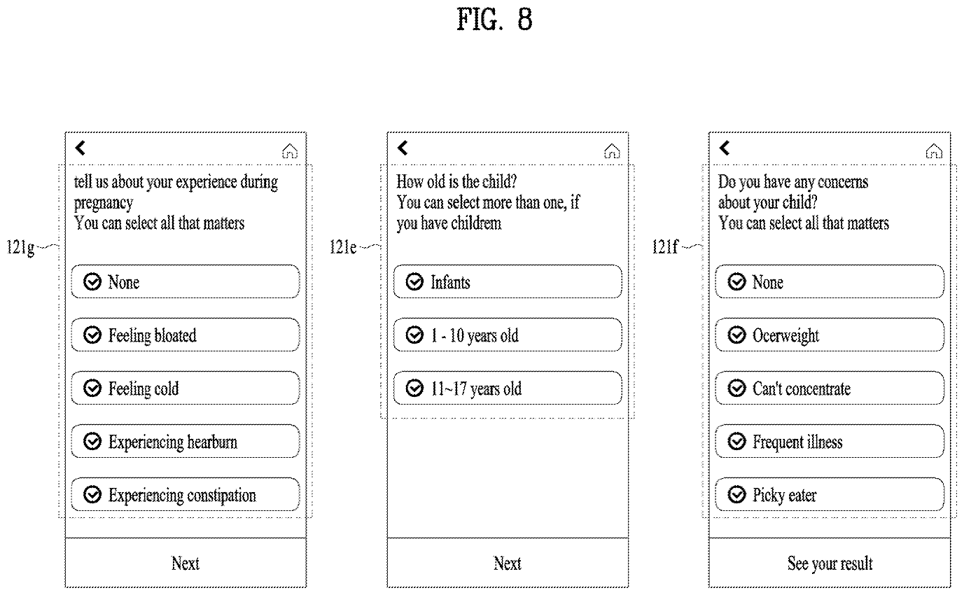

Referring to FIG. 8, the method/apparatus according to the embodiments may generate UI/UX icons indicating user's health-related experience information, family information, child information, and the like through the client and/or the interface unit, and receive user information through the UI/UX icons.

Referring to FIG. 9, the client and/or the processor of the method/apparatus according to the embodiments may analyze user information and provide health diagnosis information and/or nutritional diagnosis information to the user. The processor and/or the server may analyze nutritional supplements being consumed by the user based on the user information. For example, based on current information of the user, it is possible to analyze whether or not nutritional supplements being consumed by the user are suitable for each nutritional supplement and display analysis results. The client and/or the interface unit according to the embodiments may receive additional information from the user for detailed analysis. For example, the apparatus according to the embodiments may receive health information of interest from the user through a UI/UX icon. The client, the server, and the processor according to the embodiments may provide specific diagnostic information based on user information. Regarding specific diagnostic information, expert opinion data may be provided. For example, based on diagnostic information of experts and/or thesis information, advice information useful for the user's nutritional condition and/or health condition may be provided. Accordingly, the client, the server, the processor, and the like according to the embodiments can provide necessary diagnostic information to the user based on the user's sex, age, family relationship, child information, eating habits, and area of interest and provide the platform service required for the user.

Referring to FIGS. 6 to 9, the method/apparatus according to the embodiments may display survey items for the user on a screen, receive information input by the user, store input user survey information in the server or the database, and analyze the information. Then, the method/apparatus according to the embodiments may generate survey result information based on user survey information and display the survey result information as shown in FIG. 9. The survey result information may include information on keywords related to health in which the user is interested, information on cautions, and information on nutritional deficiencies. The information on keywords related to health in which the user is interested may be displayed as shown in the area 121k, the information on cautions may be displayed as shown in the area 121l, and information on nutritional deficiencies may be displayed as shown in the area 121m.

More specifically, the client terminal may display survey items for the user on the screen, receive user input, and transmit the same to the server or the database. The server or the database may generate survey result information based on the received user input information and transmit the survey result information to the client. The survey result information may be displayed on the screen of the client terminal based on a UI such as the areas 121k, 121l, and 121m.

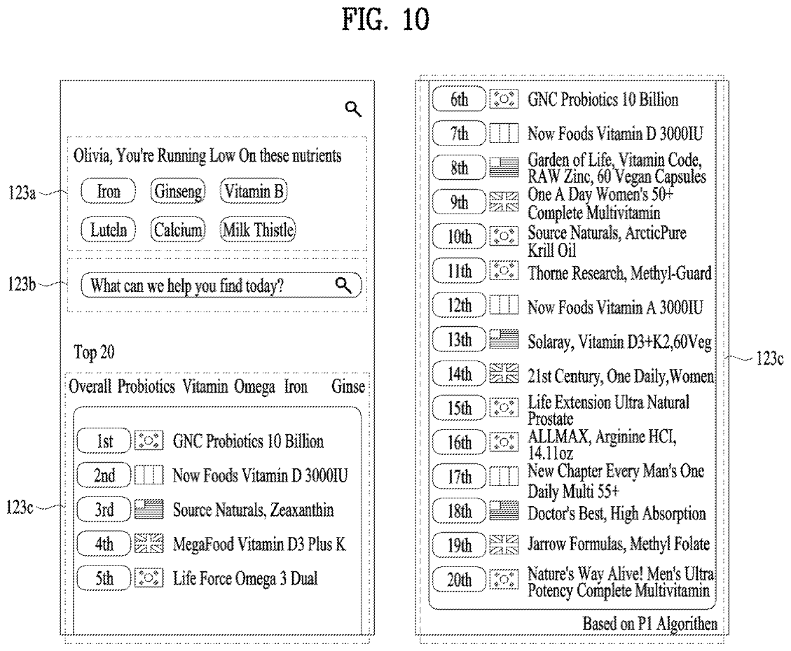

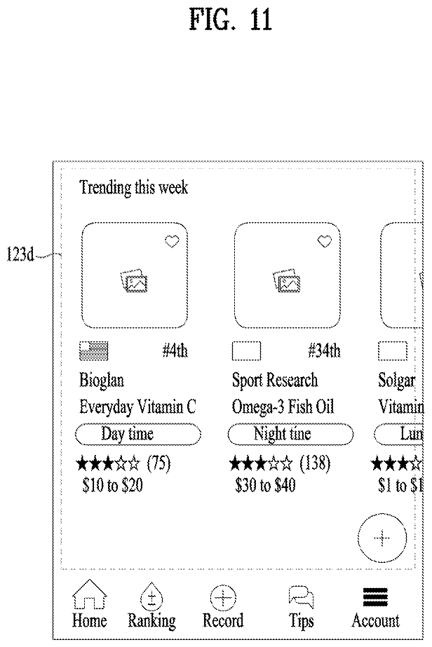

FIGS. 10 and 11 show the ranking 123 according to embodiments.

Referring to FIG. 10, the ranking 123 includes an area 123a displaying keywords for nutritional components indicating low intake levels, a search area 123b, and an area 123c displaying a ranking.

The area 123a may indicate keywords for insufficient nutrition when the user completes surveys after signing up for membership, and when the user has signed up as a member but has not yet completed surveys, display text inducing a survey (for example, “Check out the supplement combos you should never mix.”). If the user clicks on a keyword for insufficient nutrition, the user can move to a page that displays nutritional supplement products related to the keyword. When the user clicks the text inducing the survey, the user can move to the survey/results page and reply to the survey.

The area 123b is a search area and displays an input window through which the user can input a search term. The user can search for a product by directly inputting a search term into the input window.

The area 123c is an area indicating a ranking of products and may display a ranking of nutritional supplements from first to twentieth. The area 123c may display a ranking of products, countries of origin of the products, and product names together and may indicate ranking according to categories of nutritional components. For example, the ranking of products belonging to the vitamin category may be displayed. When the user clicks text listed according to the ranking, he or she can move to a detailed view screen of the nutritional supplement product of the corresponding ranking.

Referring to FIG. 11, the ranking 123 includes an area 123d displaying products whose rankings have risen sharply during the week.

The area 123d may display images of products whose rankings have risen sharply, images of countries of origin, ranking information, names, main nutritional components, recommended intake time (e.g., day, night, lunch, dinner, etc.), rating information, and price range information. Information on recommended products is arranged in the form of a card, and information on each product can be moved on the screen by a lateral scrolling operation.

Information on products whose rankings have risen sharply during the week may be registered and managed on an administrator page. That is, the client may receive information on products registered by the administrator through the server and display the information in the area 123d.

Referring to FIG. 10, the client, the server, and the processor according to the embodiments may analyze user's nutrition and health conditions based on user information stored in the memory and/or the database and provide results. Nutritional information insufficient for the user may be displayed through UI/UX icons. The client, the server, and the processor according to the embodiments may receive nutritional information capable of supplementing nutritional information insufficient for the user from the user. For example, search data may be received through a UI/UX icon indicating a search window. The client, the server, and the processor according to the embodiments may generate and display result information corresponding to nutritional information that the user wants to search from user and expert data stored in the memory and/or the database. The information generated here may be sorted based on a ranking of nutritional supplements used by many users. The client, the server, and the processor according to the embodiments may analyze nutrition and health information using an algorithm. An algorithm according to the embodiments may be referred to as a P1 algorithm. The P1 algorithm according to the embodiments may generate result data optimized for the user based on user and expert data. A frequency, an average value, a median value, and the like may be extracted based on user reviews, expert opinions, nutritional information insufficient for the user, a user's past health history, user's current eating habits, and the like, and result data may be extracted based on these statistics. In addition, weights may be assigned to data in consideration of characteristics of each user, and accuracy may be adjusted by providing a threshold.

Referring to FIG. 11, the client, the server, and the processor according to the embodiments may generate trend information for a certain period of time. According to the embodiments, analysis information, ranking information, trend information, and the like provided to the user may be generated based on data stored in the memory, the processor, the server, the database, and the like and the P1 algorithm.

The method/apparatus according to the embodiments may generate main information based on user information, process health-related information based on the user information, analyze nutritional supplement ranking information based on the user information, receive an input signal from the user, and provide recommendation and column information. Furthermore, the step of analyzing the nutritional supplement ranking information may include a step of generating the nutritional supplement ranking information based on an algorithm. Overall information of the nutritional supplement ranking information may be generated based on the algorithm, and the algorithm may analyze the overall information based on the frequency or the number of views of the user. Further, the algorithm may analyze a ranking for a specific type of nutritional supplement. The algorithm is referred to as the P1 algorithm or the like, and may be referred to as P2, P3, or the like depending on the version. Furthermore, the algorithm may analyze reliable nutritional supplement information based on production relationships between products (same company and follow-up series), sales suitability test results, sales volume, number of sales, sales period, price, advertisement, user/expert reviews, the number of resales, and the like.

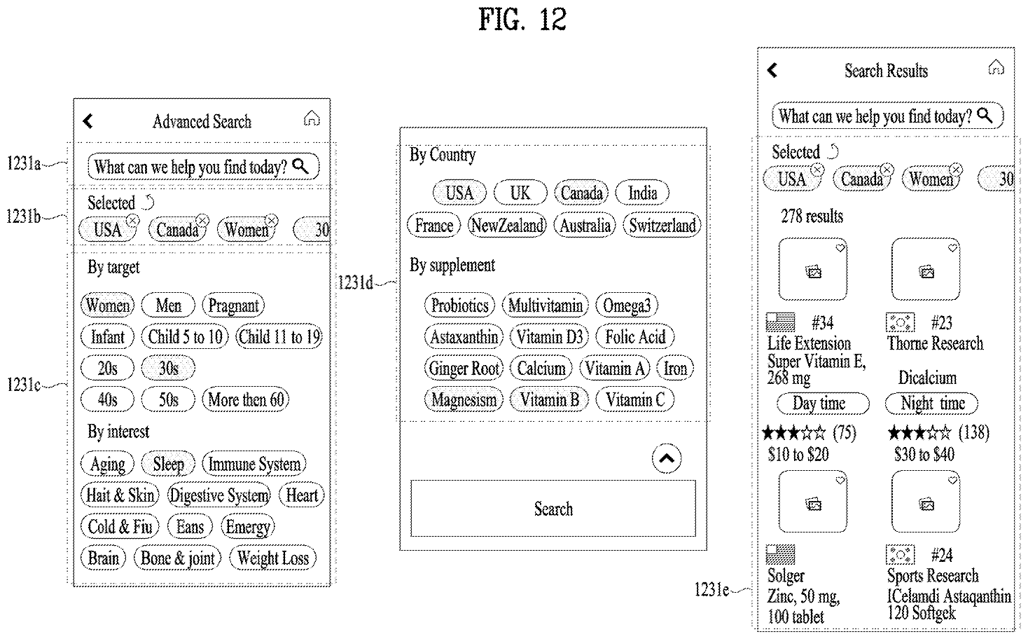

FIG. 12 shows a detailed search and result screen 1231e according to embodiments.

Referring to FIG. 12, a detailed search page may include an input window 1231a through which a search word can be directly input, an area 1231b displaying selected keywords, and areas 1231c and 1231d displaying keywords.

The input window 1231a allows the user to search fora product by directly inputting a search term.

The area 1231b is an area in which keywords selected by the user in the areas 1231c and 1231d are collected and displayed. Each selected keyword is displayed with an “x” icon, and when the user clicks the “x” icon, the selected keyword is canceled.

The areas 1231c and 1231d display various keywords for facilitating product search. The user can select the displayed keywords. Keywords may be classified and displayed according to types thereof. For example, keywords displayed under “by target” item may include only keywords related to the purpose or target of a nutritional supplement product, and keywords displayed under “by interest” item may include only keywords related to effects of a nutritional supplement product. Keywords displayed under “by country” item may only include keywords related to the country of origin of a product, and keywords displayed under “by supplement” item may include only keywords related to nutritional components of products.

When the user selects at least one keyword and clicks a “search” button, the user can move to the search result 1231e.

The area 1231e displays keywords selected by the user and lists nutritional supplement products related to the keywords. The listed nutritional supplement products may be displayed along with product images, images of countries of origin, ranking information, product names, main nutritional components, recommended intake time (e.g., day, night, lunch, dinner, etc.), rating information, and price range information. Information on recommended products is arranged in the form of a card, and information on each product may be moved on the screen by a lateral or vertical scrolling operation.

When the user clicks a product listed in the area 1231e, the user can move to the product details 1232. The method/apparatus according to the embodiments may display the product details 1232 in response to an operation of clicking on a product listed in the area 1231e.

FIG. 12 shows a process in which the client, the server, and the processor according to the embodiments receive information from a user and process result data in response to the received information. The client, the server, and the processor according to the embodiments may provide an advanced search process to the user. The client, the server, and the processor according to the embodiments may generate a UI/UX icon through which the user can input information for search, receive an input selection signal from the user, receive target information from the user, and receive interest information, country information and nutritional supplement information. The client, the server, and the processor according to the embodiments may accurately and rapidly display result information corresponding to user selection based on information received from the user, data stored in the memory and/or the database, and the P1 algorithm.

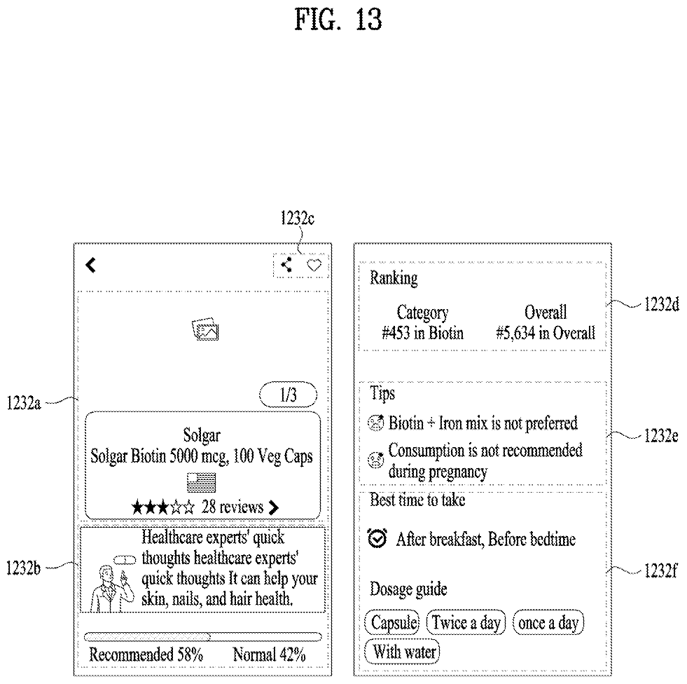

FIGS. 13, 14 and 15 show the product details 1232 according to embodiments.

Referring to FIG. 13, the product details 1232 include an area 1232a displaying basic product information, an area 1232b displaying one-line summary information and recommendation/normal index information regarding a product, and an area 1232c displaying a sharing/favorite icon. In addition, the product details 1232 may include an area 1232d displaying rankings of a corresponding product, an area 1232e displaying tips related to intake of the corresponding product, and an area 1232f for guiding a desired intake time and intake amount of the corresponding product.

The area 1232a displays a product image, a product name, an origin flag image of the product, star rating information, and the number of reviews. A plurality of product images may be displayed, and the next image may be displayed by a scrolling operation. The method/apparatus according to the embodiments may display a review screen for a corresponding product in response to an operation of clicking on text representing the number of reviews.

The area 1232b displays one-line information about the product. For example, information about the product, such as “It can help your skin, nails, and hair health.” can be displayed in the area 1232b. In addition, a recommended/normal index may be displayed in a bar form therein. Recommended/normal index and one-line information may be registered and managed by the administrator.

The area 1232c is located at the top of the screen and displays a share icon and a heart icon. The user can share information about the corresponding product with others using another SNS application through the share icon and display products of interest to the user through the heart icon.

The area 1232d displays rankings of the corresponding product. The area 1232d may display both a ranking within a category in which the corresponding product is classified and an overall ranking. The overall ranking and the category ranking may be registered and managed by the administrator.

The area 1232e may display tips useful for the user when taking the corresponding product as text. For example, the area 1232e may display nutritional components that are undesirable if the user takes together with the corresponding product or display cautions, such as “Biotin+Iron mix is not preferred” and “Consumption is not recommended during pregnancy.”

The area 1232f is an area for displaying items related to intake of the corresponding product. Information on a best time to take the corresponding product and information on an intake amount may be displayed.

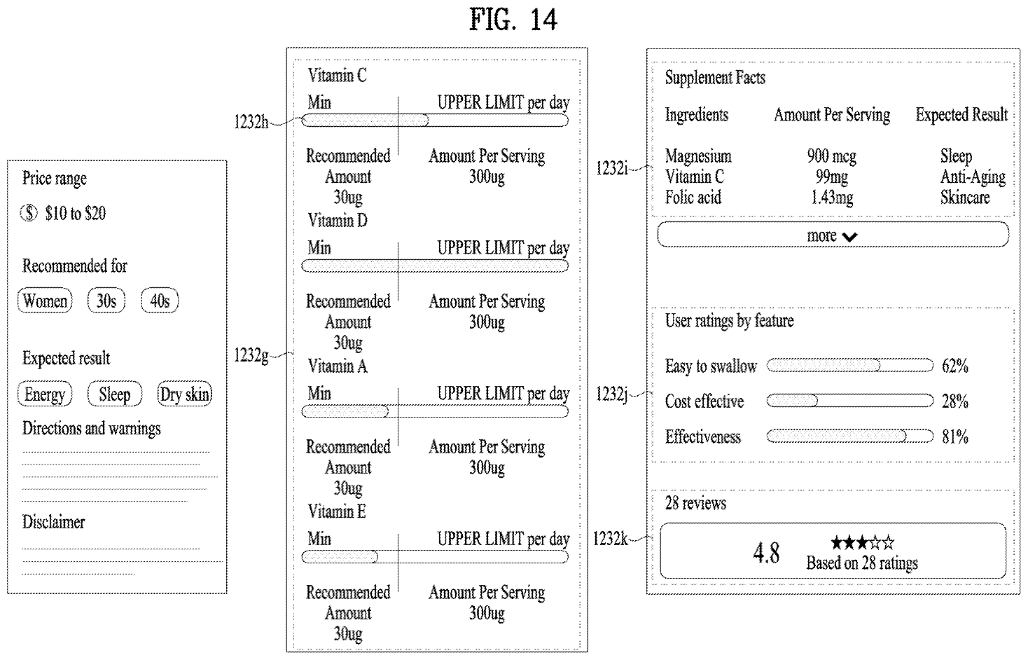

Referring to FIG. 14, the product details 1232 may include an area displaying information on a product price, keyword information (e.g., “female”, “30s”, “40s”, etc.) on a target for which the product is recommended, and keyword information (e.g., “energy”, “sleep”, “dry skin”, etc.) on product effects. In addition, an area displaying cautions on the product may be included.

In addition, the product details 1232 may include an area 1232g displaying the component content of the product, an area 1232i displaying nutritional component analysis, an area 1232j displaying user rating, and an area 1232k displaying star rating information.

The area 1232g displays the nutritional content of the product and a recommended daily amount using a bar graph. If the nutritional content is higher than a maximum recommended daily amount, the color of the entire bar is displayed in red, and if not, the color of a part of the gray bar which corresponds to the nutritional content is displayed in green. The bar graph is displayed for each nutritional component, and if there are multiple nutritional components, only bar graphs for four main nutritional components may be displayed.

The area 1232i displays the names, content, and expected effects of nutritional components.

The area 1232j displays results of ratings by a plurality of users for predetermined review items for product evaluation. For example, rating results of users for items such as “easy to swallow,” “cost effective” and “effectiveness” may be displayed. Rating results of users may be displayed as bar graphs. For example, if 62% of all users select the item “easy to swallow,” the bar graph of the item may represent 62%.

The area 1232k is an area displaying the number of reviews and star rating information on the product. The star rating information is information with a perfect score of five stars and may be star rating information obtained by averaging information evaluated by users. The star rating information may also indicate the number of users who participated in evaluation.

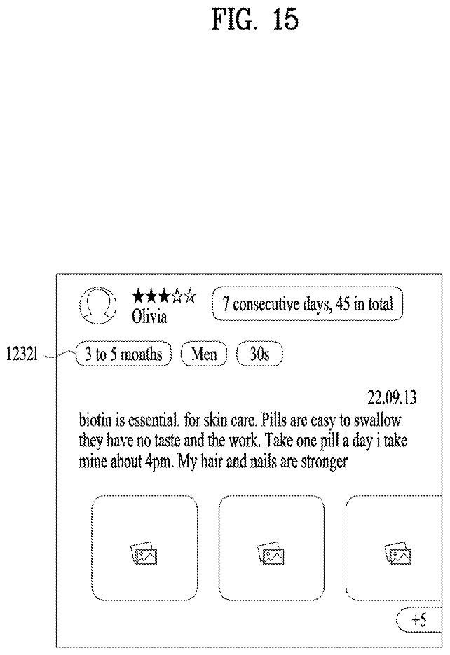

Referring to FIG. 15, an area 12321 indicating review information created by a user is displayed.

The product details 1232 may include the area 12311.

The area 12311 may display an image of a user, star rating information registered by the user, keyword information related to the user, text directly written by the user, and a product image registered by the user. In the area 12321, star point information and user nickname may be displayed together next to the user image, and a user's current challenge progress may also be displayed. In addition, keywords related to the user may include a period during which the user has consumed the corresponding product, the sex and age group of the user, and the like.

The product details 1232 may include a “write your review” button at the bottom, and the method/apparatus according to the embodiments may display a screen for writing a review in response to clicking on the “write your review” button.

Referring to FIG. 13, the client, the server, and the processor according to the embodiments may provide optimized nutritional intake recommendation information to a user. The generated information may include nutritional supplement information, components, user reviews, ranking information, and the like. The client, the server, and the processor according to the embodiments may analyze analysis information of a user and generate diagnostic information of experts. Whether result information is suitable for the user may be represented through UI/UX icons such as a graph and/or a percentage. Accordingly, the user can receive feedback opinions of experts and use accurate and reliable health care platform services. The client, the server, and the processor according to the embodiments can provide users with reliable and verified diagnosis and recommendation information from a number of experts and users instead of opinions of individual users, opinions of sellers, and the like. The user can be provided with nutritional supplement components, a recommended intake time and method, expert eating habit recommendations, and the like.

Referring to FIG. 14, the client, the server, and the processor according to the embodiments may provide a detailed procedure of the above-described method of providing diagnostic recommendation information. The client, the server, and the processor according to the embodiments may receive price information of nutritional products through a network and generate price range and/or average price information. By analyzing information on nutritional products, it is possible to generate information on a sex and ages for which intake is recommended, expected effects, and expert opinions. In addition, nutritional component content and daily recommended amount may be represented using UI/UX icons that are easy to identify. The user can accurately and conveniently ascertain insufficient nutritional information for each nutritional component through the information generated by the embodiments. In addition, the client, the server, and the processor according to the embodiments can generate effects for each component of nutritional supplements, notification information for users, intake information, review information, scores, and the like and display UI/UX icons corresponding to the information.

Referring to FIG. 15, the client, the server, and the processor according to the embodiments may receive detailed selection signals from a user, generate more detailed effects of each component of a nutritional supplement, notification information for the user, intake information, review information, scores, and the like and display UI/UX icons corresponding to the information.

Accordingly, the client, the server, and the processor according to the embodiments can accumulate usage data of the user in a database and generate more accurate diagnosis and recommendation information for subsequent users. The usage data accumulated in the database can be used to train the P1 algorithm and improve suitability.

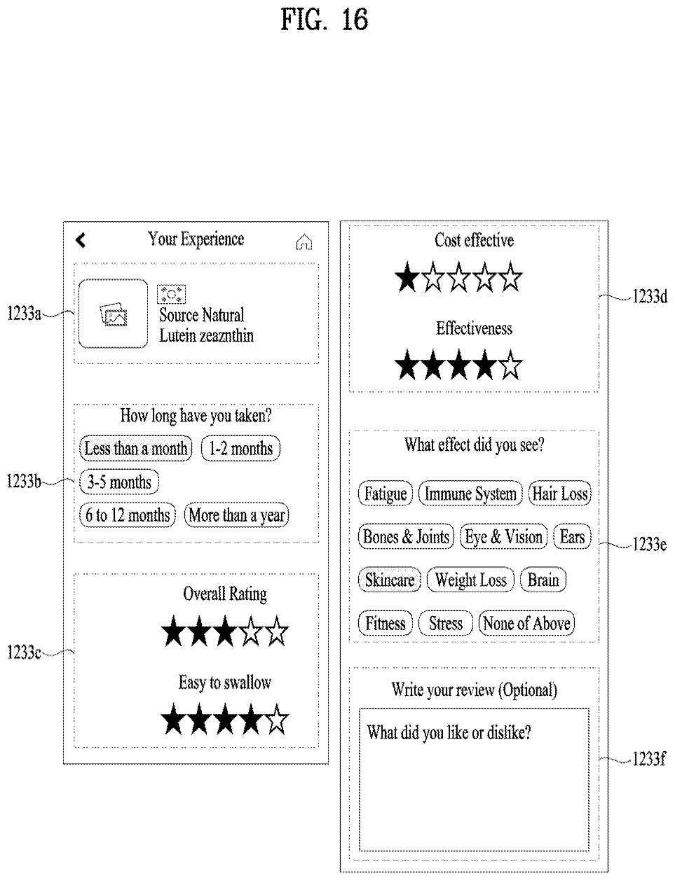

FIG. 16 shows the writing review 1233 according to embodiments.

Referring to FIG. 16, the writing review 1233 may include an area 1233a displaying an image and name of a product to be reviewed, an area 1233b for selecting a keyword indicating the intake period of the product, areas 1233c and 1233d for star rating for each item, an area 1233e for selecting a keyword representing an effect, and an input window 1233f for writing a review.

The area 1233a is an area displaying an image of a product to be reviewed, an image of the country of origin, and a product name.

The area 1233b displays keywords representing intake periods of the corresponding product. For example, the keywords may include keywords such as “less than a month,” “1-2 months,” “3-5 months,” “6-12 months,” and “more than a year.” The user may select an intake period for the corresponding product by selecting one of the displayed keywords.

The areas 1233c and 1233d are areas for determining star scores for predetermined items. Five stars are displayed for items such as overall rating, “easy to swallow,” “cost effective,” and effectiveness, and the user can select a predetermined number of stars among the five stars to score. Depending on user's selection for each item, how many stars are selected out of the five stars may be displayed. The content and number of items may be changed by the administrator.

The area 1233e displays keywords for product effects. At least one of a plurality of keywords may be selected by the user. Selected keywords may change color to indicate selection. Keywords may include “fatigue,” “immune system,” “hair loss,” “bone,” “joints,” “eye,” “ears,” “skincare,” “weight loss,” “brain,” “fitness,” “stress,” “none of above,” and the like.

The area 1233f is an input window in which the user directly writes a review.

Keywords or content selected by the user may be displayed in the area 12321 of the product details 1232 indicating review information.

FIG. 16 shows an interface UI/UX through which the client, the server, and the processor according to the embodiments receive information from a user. The client, the server, and the processor according to the embodiment generate UI/UX for user convenience through an interface unit and receive a selection signal of the user. Accordingly, the client, the server, and the processor according to the embodiments may accumulate usage data of the user in a database and generate more accurate diagnosis and recommendation information for subsequent users. The usage data accumulated in the database may be used to train the P1 algorithm and improve suitability.

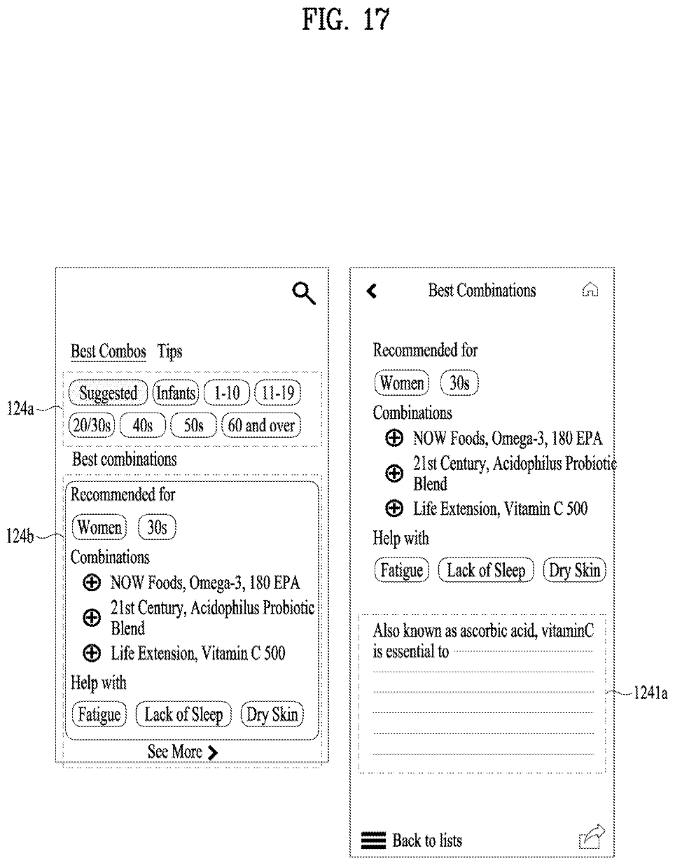

FIG. 17 shows best combinations 124 and a detailed view screen according to embodiments.

Referring to FIG. 17, the best combinations 124 may include an area 124a displaying keywords related to combinations and an area 124b displaying combination information.

The area 124a displays keywords for targets for which nutritional supplement combinations are recommended. For example, keywords such as “suggested,” “infants,” “1-10,” “11-19,” “20/30s,” “40s,” “50s,” “60 and over,” and the like may be displayed. Selected keywords may be displayed in a different color from the others to indicate selection.

The area 124b displays combination information on products that are good to take together based on results of surveys for the user. The area 124b may display keywords (e.g., “female” and “30s”) for a target for which combined products are recommended and display keywords (e.g., “fatigue,” “lack of sleep,” and “dry skin”) related to information on the combined products and symptoms that can be improved by the combined products.

The user can move to the detailed view screen by clicking a “see more” button in the area 124b. The detailed view screen includes an area 1241a displaying description of the corresponding product combination.

Referring to FIG. 17, the client, the server, and the processor according to the embodiments may generate recommended combination information optimized for a user. In order to recommend accurate combination information to the user, categories may be determined for each keyword. The categories may include the age, family information, and the like of the user. The client, the server, and the processor according to the embodiments may provide nutritional and health recommendation combination information that meets characteristics of the user based on data accumulated in the database. In addition, expert opinions related to recommendation combination information for the user may be filtered and generated as detailed description information. Accordingly, there is an effect that the user can efficiently use reliable nutrition and health recommendation information based on data accumulated from a plurality of users and experts.

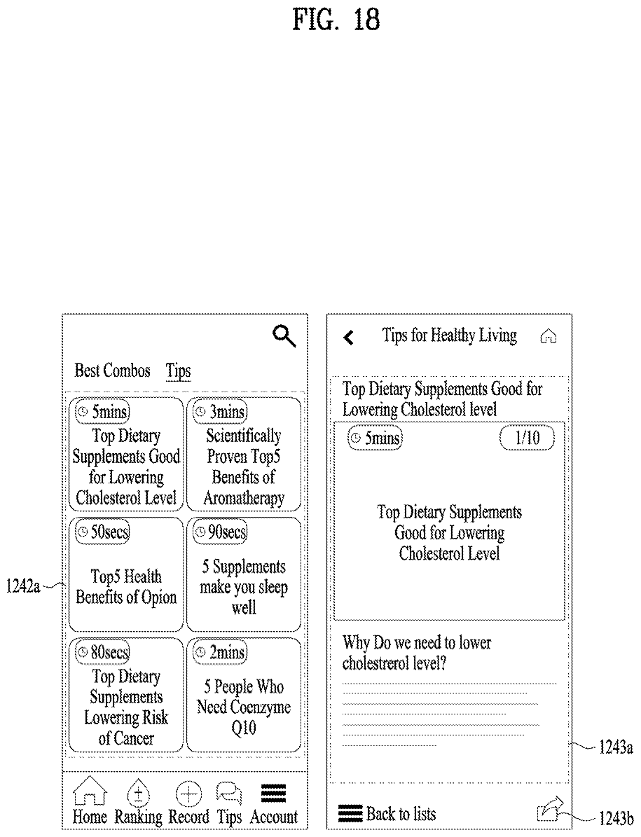

FIG. 18 shows a pharmacist column list screen and a detailed view screen according to embodiments.

Referring to FIG. 18, the pharmacist column list screen includes an area 1242a listing columns written by pharmacists.

The area 1242a lists box areas in which the titles of columns are displayed. The user may click on one of the listed box areas to move to a detailed view screen of the corresponding column.

The detailed view screen of the column includes an area 1243a. The area 1243a may display the title, image and contents of the column. The detailed view screen displays a share icon 1243b, and when the user clicks the share icon 1243b, the column can be transmitted using another application (SNS, e-mail, cloud service, text message software, or the like).

Referring to FIG. 18, the client, the server, and the processor according to the embodiments may receive and store expert opinions from experts through the interface unit and the database. Furthermore, it is possible to improve the accuracy of information by learning professional opinion information using the P1 algorithm. In addition, it is possible to provide the user with expert information corresponding to nutrition necessary for the user in the form of UI/UX by searching the database for the expert information.

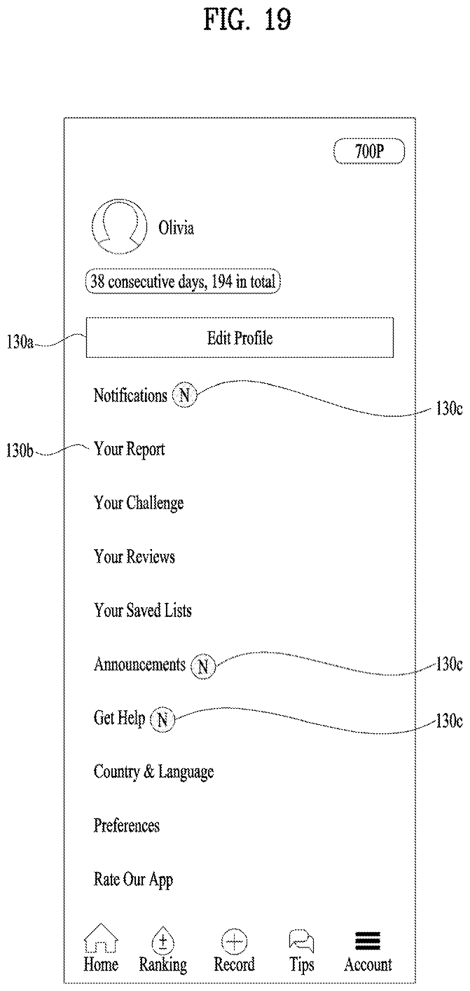

FIG. 19 shows the My Page 130 screen according to embodiments.

Referring to FIG. 19, the My Page 130 displays a button 130a for editing a user's profile and items for direct navigation to various pages. For example, the items may include Notification, Your Report 130b, Your Challenge, Your Reviews, Your Saved Lists, Announcements, Get Help, Country & Language, Preferences, Rate Our App, and the like. When new content is uploaded to a page linked from each item, an icon 130c for notifying the user thereof may be displayed next to the corresponding item. The 130c icon may disappear when the user checks the uploaded content.

The user may move to the survey/result 121 by clicking the item “Your Report.” In addition, the user can move to the challenge 131 by clicking the item “Your Challenge.” The challenge 131 may be a page including the areas 120b and 120c of FIG. 3. The user can move to the review 132 displaying a collection of reviews written by users through the item “Your Reviews.” The user can move to the saved product list 133 listing products saved by the user through the item “Your Saved Lists.”

The types and order of items displayed on My Page 130 may be changed by the administrator.

Referring to FIG. 19, the client, the server, and the processor according to the embodiments may generate UI/UX menu information for helping a user use the platform and improving convenience and accessibility of the platform. The UI/UX menu information is not fixed according to system settings, but may be composed of information for providing information necessary and recommended for a user based on user information.

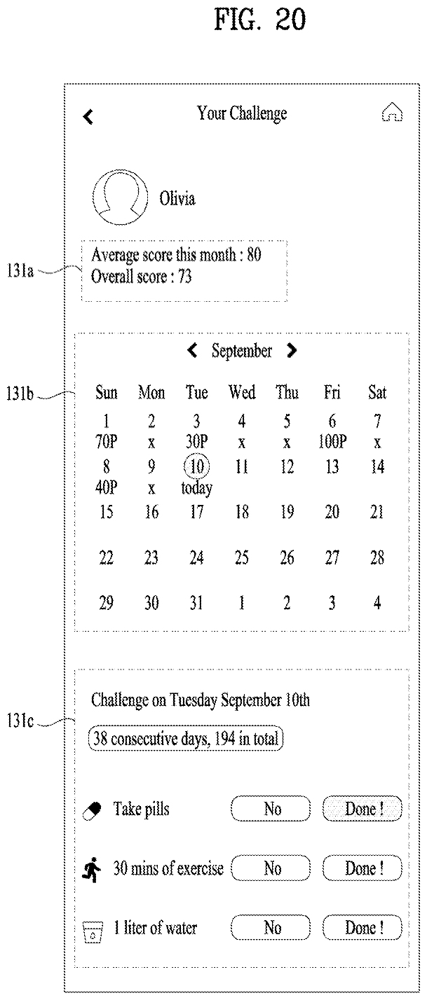

FIG. 20 shows the challenge 131 according to embodiments.

Referring to FIG. 20, the challenge 131 includes an area displaying an image and nickname of a user, and an area 131a displaying points obtained by the user through challenges. In addition, the challenge 131 may include an area 131b displaying a calendar and an area 131c for recording whether a challenge has been performed.

The area 131a displays points obtained by the user by performing the challenge in various ways. For example, the average score per month may be displayed, or the overall score may be additionally displayed.

The area 131b may display a calendar and points obtained by the user for each date indicated on the calendar.

The area 131c displays the current date and each challenge item, and displays buttons (“No” button and “Done” button) for indicating whether each challenge item has been performed. The user may click the “No” button or the “Done” button depending on whether the challenge has been performed and obtain points based on results of challenge performance of the user. Point calculation according to performance of the challenge by the user may be performed in the server, and the record of performance of the challenge may be stored in the database.

Referring to FIG. 20, the client, the server, and the processor according to the embodiments may generate challenge UI/UX information for recommending use of the platform and inducing healthy habits. By creating UI/UX menu information that provides such convenience and accessibility, the user can pursue a continuous healthy lifestyle while using reliable platform information.

The method/apparatus (e.g., the client) according to the embodiments may receive information on whether a user performs a challenge item and transmit the information to the server or the database. The server or the database according to the embodiments may create/store user point information based on the received information and transmit the point information to the client. The client may display the received point information on the screen.

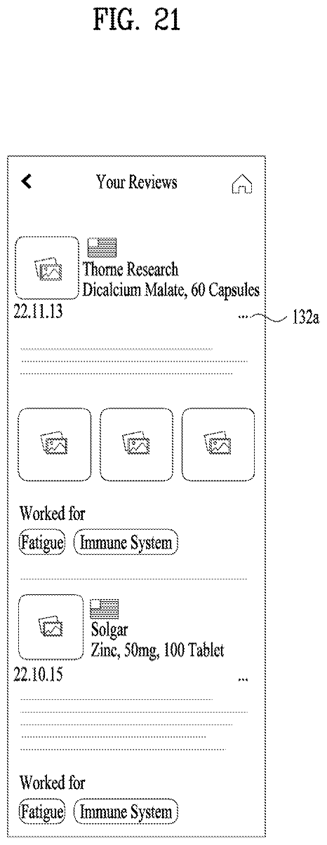

FIG. 21 shows the review 132 and the save product list 133 according to embodiments.

The review 132 may collect and display reviews written by users. Each review area may be displayed like the area 12321 for displaying a review shown in FIG. 15. Reviews of products written by users may be listed in the review 132.

The saved product list 133 may collect and display information on products saved by the user. Information on saved products may be generated and listed in the form of a card for each product. Each card area includes a heart icon 133a, and when the user clicks the icon 133a, the corresponding product may be excluded from the list. Each card area may include a product image, an image of the country of origin of the corresponding product, a product name, intake time periods of the product, star rating information, and price range information.

Referring to FIG. 21, the client, the server, and the processor according to the embodiments may generate history data for helping a user use the platform as UI/UX information. Past review data of users may be received from the database in response to a selection signal of a user, and a saved list of the user may be received in response to a selection signal of the user. Here, received data may be converted into UI/UX information representing the same and displayed.

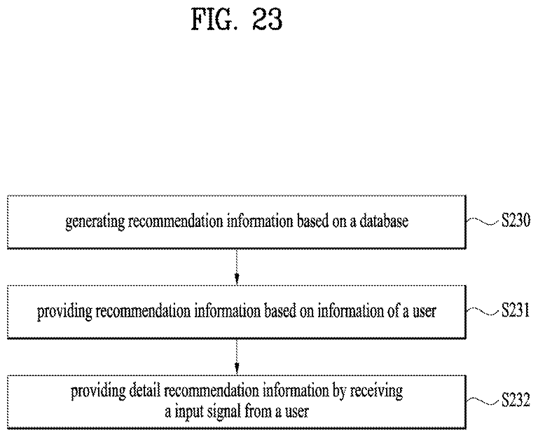

FIG. 23 illustrates a method for an online prescription platform according to embodiments.

FIG. 23 shows a method for the online prescription platform, and specifically, a method of providing optimal recommendation combination information to a user. The recommendation combination information may correspond to the information 124 shown in FIG. 1, the information 120e shown in FIG. 4, the best recommendation information shown in FIGS. 17 and 18, and the like. The method for the online prescription platform may be performed by the client 250 and the server 251 of FIG. 25. Further, the method for the online prescription platform may be performed by the client 260, the server 261, and the like shown in FIGS. 26A and 26B.

The method for the online prescription platform according to the embodiments may include step S230 of generating recommendation information based on a database.

The method for the online prescription platform according to the embodiments may generate recommendation information through a database connected to the client, the server, or the like. The method for the online prescription platform according to the embodiments may generate recommendation information suitable for a current user through data accumulated in the database. Here, the generated information may be displayed as UI/UX information as shown in the drawings described above. A process of generating and processing recommendation information may be performed by the P1 algorithm.

The method for the online prescription platform according to the embodiments may further include step S231 of providing recommendation information based on user information.

The method for the online prescription platform according to the embodiments may provide recommendation information based on user information stored in a memory and/or the database. Further, information may be received from a user through the interface unit of the client and/or the server. In the method for the online prescription platform according to the embodiments, recommendation information suitable for a situation of the current user may be generated based on information received from the database and information additionally received from the user in step S230. Here, the generated information may be displayed as UI/UX information as shown in the drawings described above. A process of generating and processing recommendation information may be performed by the P1 algorithm.

The method for the online prescription platform according to the embodiments may further include step S232 of providing detailed recommendation information by receiving an input signal from the user.

The method for the online prescription platform according to the embodiments may receive an additional input signal through the interface unit in order to provide a platform health recommendation service optimized for the user. For example, survey information such as the survey information 121 shown in FIG. 1, the survey information shown in FIGS. 6 to 10 and 12 to 21, and the like may be received. Based on survey information received from the user, specific recommendation information may be generated. Here, the generated information may be displayed as UI/UX information as shown in the drawings described above. A process of generating and processing recommendation information may be performed by the P1 algorithm.

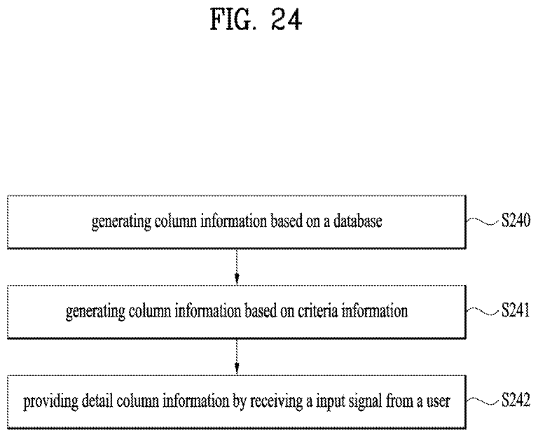

FIG. 24 illustrates a method for an online prescription platform according to embodiments.

FIG. 24 shows the method for the online prescribing platform, and specifically, a method of providing a user with accurate opinions and diagnostic information of experts. The opinions/diagnostic information may correspond to expert opinion information shown in FIG. 9, FIG. 13, FIG. 14, FIG. 17, and FIG. 18, and the like. The method for the online prescription platform may be performed by the client 250 and the server 251 shown in FIG. 25. Further, the method for the online prescription platform may be performed by the client 260, the server 261, or the like shown in FIGS. 26A and 26B.

The method for the online prescription platform according to the embodiments may include step S240 of generating column information based on a database.

The method for the online prescription platform according to the embodiments may receive expert knowledge information from experts through the interface unit of the client and/or the server. The method for the online prescription platform according to the embodiments may generate expert column information (expert diagnostic opinion information) based on data stored in the database in response to a selection signal of a user. Here, the generated information may be displayed as UI/UX information as shown in the drawings described above. A process of generating and processing recommendation information may be performed by the P1 algorithm.

The method for the online prescription platform according to the embodiments may further include step S241 of providing column information based on criteria information.

The method for the online prescription platform according to the embodiments may search for, filter, and process expert column information based on user information stored in the memory and/or the database. The criteria information may be information about characteristics, environment, and health condition of the user. Information may be received from the user through the interface unit as described above, and expert information optimized for the user may be generated based on the received and stored information. Here, the generated information may be displayed as UI/UX information as shown in the drawings described above. A process of generating and processing recommendation information may be performed by the P1 algorithm.

The method for the online prescription platform according to the embodiments may further include step S242 of providing detailed column information by receiving an input signal from the user.

The method for the online prescription platform according to the embodiments may receive an input signal from the user and generate detailed professional information more suitable for user information. A selection signal of the user for a category of information may be received, and result information (expert opinion information) corresponding to the selection signal may be generated and provided to the user. Here, the generated information may be displayed as UI/UX information as shown in the drawings described above. A process of generating and processing recommendation information may be performed by the P1 algorithm.

FIG. 25 shows an apparatus for an online prescription platform according to embodiments.

The structure of the apparatus for the online prescription platform according to the embodiments is as shown in FIG. 25. A system related to the apparatus for the online prescription platform according to the embodiments may include the client 250, the network 252, the server 251, and/or the database 253. Each component constituting the system may correspond to hardware, software, a processor, and/or a combination thereof.

The client 250 may perform the method for the online prescription platform described above. The client may receive information from a user and/or experts, provide necessary information to the user based on the received information, and generate UI/UX information with high convenience in order to provide the information. The client may be a client machine or may be a client application implemented in a mobile device, a PC, or the like.

The network 252 may connect the client and the server. The client may store information received from the user and recommendation result information provided to the user in the memory of the client, and may transmit the information to the server and/or the database through the network. The network may be a broadband network such as 5G or 6G, the Internet, or the like. The server may store information received from the user and recommendation result information provided to the user in the memory of the server, and may transmit the information to the client and/or the database through the network.

The server 251 is a server corresponding to a client, and may be a server machine and/or a server application. Like the client, the server may perform the operation of the client. Results of operation performed by the server may be transmitted to the client through the network. The server may perform the method for the online prescription platform.

The database 253 may store information necessary for operations of the client and/or the server, information generated as a result of an operation, and information corresponding to signals received from users and/or experts. The database 253 may provide information to the server or the client. Information stored in the database and memories may be trained through the P1 algorithm.

FIG. 25 shows an apparatus according to embodiments. The apparatus according to the embodiments includes the client 250, the server 251, or the database 253. Each component may be functionally separated. The client 250 may be a terminal capable of communication used by a user. For example, the client 250 may include electronic devices capable of computation, storage, transmission/reception, and display of information such as a smartphone, a mobile phone, a desktop computer, and a laptop computer.

The client 250 may include an application for providing the platform service. The application may provide a user interface for a user to input information or display received information and transmit information. The client 250 displays information received from the server according to a UI format defined by the application, displays a different screen according to a user operation, or transmits information to the server 251 or the database 253 through the network 252. A user using the platform service according to the embodiments may be a general consumer who seeks information on nutritional supplements or an expert (pharmacist, doctor, or product manufacturer) who provides information on nutritional supplements.

The client 250 may include a camera module. The client 250 may allow an application to take a picture using the camera module and may transmit information on a captured image to the server or the database. In this case, the image information may be a photograph of a nutritional supplement or a label thereof.

The client 250 may include a GPS module. The client 250 may allow an application to display the location of the client 250 using the GPS module and may display the location of the client 250 and locations of nearby pharmacies (or pharmacists working at the pharmacies). The client 250 may transmit location information of the client 250 to the server 251 or the database 253, and the server 251 or the database 253 may transmit information on nearby pharmacies to the client 250 based on the received location information.

The server 251 may transmit/receive information to/from the client 250 or the database 253, store/calculate received information, and generate information. In addition, the server 251 may receive information from the administrator in order to provide the platform service. The administrator may input information on nutritional supplement combinations, ranking information, pharmacist columns, recommended nutritional supplement information, and pharmacy information into the server.

The server 251 may receive image information of the label of a nutritional supplement from the client 250 and search for information on the nutritional supplement based on the image information. For example, the server 251 may search nutritional supplement information stored in the server 251 for information matching the captured nutritional supplement and transmit the information on the captured nutritional supplement to the client 250. The server 251 may include a recognition module that identifies letters or numbers from image information. When the recognition module identifies letters or numbers from image information, the server 251 may search for nutritional information based on the identified information and transmit the retrieved information to the client 250.

The server 251 may receive location information from the client 250 and search for information on pharmacies located close to the client 250 based on the location information. For example, the server 251 may search location information of pharmacies stored in the server 251 for information on pharmacies located close to the location corresponding to the received location information of the client and transmit the information on the pharmacies to the client 250.

The database 253 may transmit/receive information to/from the client 250 or the server 251, store/calculate received information, and generate information. In addition, the database 253 may receive information from the administrator in order to provide the platform service. The administrator may input information on nutritional supplement combinations, ranking information, pharmacist columns, recommended nutritional supplement information, or pharmacy information into the database.

The database 253 stores information on nutritional supplements, and may transmit information on specific nutritional supplements to the server upon request from the server 251. For example, the server 251 may request nutritional supplement information from the database 253 based on information identified from an image, and the database 253 may search for nutritional supplement information matching information received from the server 251 and transmit the same to the server 251.

The database 253 may receive location information from the client 250 and search information on pharmacies located close to the client 250 based on the location information. For example, the database 253 may search information on locations of pharmacies stored in the database 253 for information on pharmacies located close to the location corresponding to the received location information of the client and transmit the corresponding pharmacy information to the client 250. Alternatively, the database 253 may search information on pharmacies located close to the client 250 and transmit corresponding pharmacy information to the server 251 at the request of the server 251.

The online platform service according to embodiments may be provided by organic operations of apparatuses according to embodiments.

FIGS. 26A and 26B show an apparatus for the online prescription platform according to embodiments.

FIGS. 26A and 26B show an apparatus for the online prescription platform according to embodiments, and specifically show the client 260 and the server 261. The client and the server in FIGS. 26A and 26B correspond to the client and the server in FIG. 25.

The client 260 may include an interface unit 2600, a memory 2601, and/or a processor 2602. The interface unit 2600 may receive information from users and/or experts. The interface unit may store received information in the memory or transmit the same to the processor. The memory may store information received through the interface unit and transmit necessary information to the processor. The memory may store instructions for performing the online prescription platform method of the processor. The processor may perform the online prescription platform method using the instructions for performing the online prescription platform method stored in the memory. The instructions stored in the memory may cause the processor to perform the online prescription method according to the above-described embodiments.

The server 261 may include an interface unit 2610, a memory 2611, and/or a processor 2612. The interface unit 2610 may receive information from users and/or experts. The interface unit may store received information in the memory or transmit the same to the processor. The memory may store information received through the interface unit and transmit necessary information to the processor. The memory may store instructions for performing the online prescription platform method of the processor. The processor may perform the online prescription platform method using the instructions for performing the online prescription platform method stored in the memory. The instructions stored in the memory may cause the processor to perform the online prescription method according to the above-described embodiments. The online prescription platform method according to embodiments may be performed by the client and/or the server.

Referring to FIGS. 26A and 26B, the interface unit 2600 or 2610 of the client 260 or the server 261 may be an application that provides a UI/UX environment for interaction between a user or the administrator and the apparatus. The processor may perform operations or instructions for driving the interface unit.

The apparatus for providing the online platform service includes a processor and a memory storing instructions of the processor. The processor may be connected to the memory and operate based on instructions stored in the memory. For example, the apparatus for providing the online platform service may be the server shown in FIG. 25 or FIGS. 26A and 26B.

Referring to FIGS. 26A, 26B and the aforementioned drawings, the processor according to the embodiments may receive user survey information, generate survey result information based on the user survey information, and transmit the generated survey result information to a client. The survey information may include information indicating the age of a user, information indicating nutritional components consumed by the user, or information indicating the sex of the user. The survey result information may include information indicating cautions on nutritional components to be consumed and information indicating insufficient nutritional components.

Further, the processor according to the embodiments may generate recommendation information based on user survey information and transmit the generated recommendation information to a client. The recommendation information may include information representing a combination of products. Further, the recommendation information may include information representing keywords related to symptoms that can be improved by products.

The processor according to the embodiments may generate challenge information based on user survey information and transmit the challenge information to a client according to embodiments. Further, the processor may receive action information (or performance information) of a user for the challenge information. The challenge information represents a challenge including at least one of taking medicine, exercising, or drinking water. Action information may indicate whether a challenge is performed.

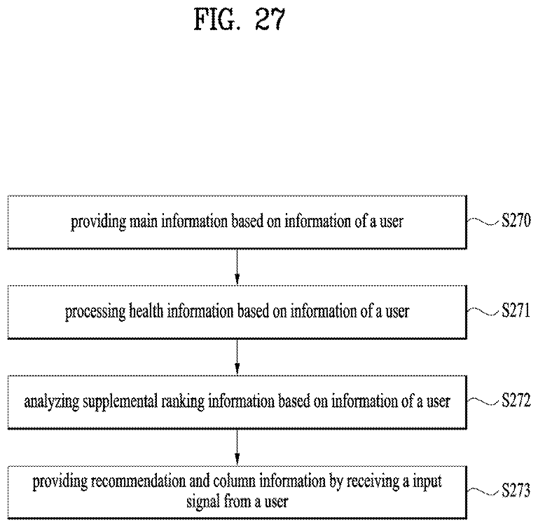

FIG. 27 illustrates a method for the online prescription platform according to embodiments.

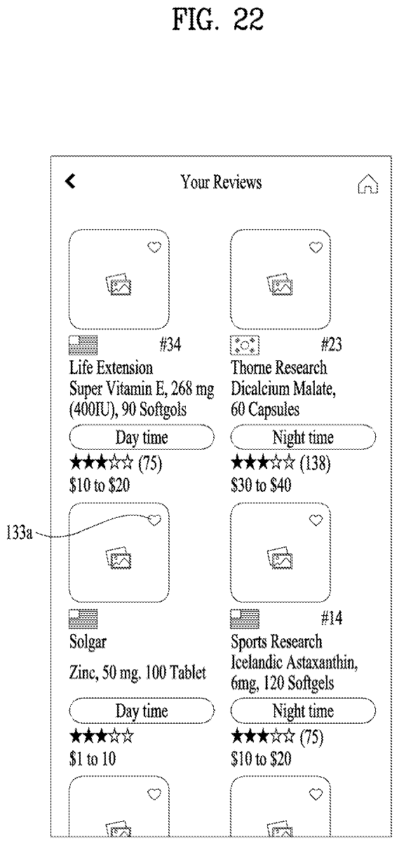

FIG. 27 shows a method performed by the apparatus shown in FIG. 25 and/or FIGS. 26A and 26B. The method of FIG. 27 may include the method of FIGS. 1 to 24.

The method for the online prescription platform according to the embodiments may include step S270 of providing main information based on user information.

The method for the online prescription platform according to the embodiments may receive user information, generate menu information based on the user information, and display the menu information. The method for the online prescription platform according to the embodiments may display preset main information according to settings of a platform system.

The method for the online prescription platform according to the embodiments may further include step S271 of processing health information based on the user information.

The method for the online prescription platform according to the embodiments may receive the user information and process health information stored in a memory and/or a database based on a criterion corresponding to the received user information. Health information necessary for the user may be received from the memory and/or the database. In order to generate the health information necessary for the user, data stored in the memory and/or the database may be further processed through the P1 algorithm.

The method for the online prescription platform according to the embodiments may further include step S272 of analyzing nutritional supplement ranking information based on the user information.

The method for the online prescription platform according to the embodiments may analyze nutritional supplement ranking information based on the user information. Data stored in the memory and/or the database may be analyzed, and criteria for analysis may be set based on the user information.

The method for the online prescription platform according to the embodiments may further include step S273 of receiving an input signal from a user and providing recommendation information and column information.

The method for the online prescription platform according to the embodiments may receive an input signal from the user and generate recommendation information and/or column information corresponding to information corresponding to the input signal. Expert information may be received from an expert through the interface unit, and data stored in the memory and/or the database may be utilized. The recommendation information and/or the column information may be displayed as accurate and highly convenient UI/UX icons according to characteristics of the user.

The method of providing the online platform service according to the embodiments may correspond to the operation performed by the apparatus according to the embodiments described with reference to FIGS. 26A and 26B. The method of providing the online platform service may be performed by the server shown in FIG. 25 or FIGS. 26A and 26B.

The method according to the embodiments may receive user survey information, generate survey result information based on the user survey information, and transmit the generated survey result information to a client. The survey information may include information indicating the age of the user, information indicating nutritional components consumed by the user, or information indicating the sex of the user. The survey result information may include information indicating cautions on nutritional components to be consumed and information indicating insufficient nutritional components.

Further, the method according to the embodiments may generate recommendation information based on the user survey information and transmit the generated recommendation information to the client. The recommendation information includes information representing a combination of products. Further, the recommendation information includes information representing keywords related to symptoms that can be improved by products.

The method according to the embodiments may generate challenge information based on the user survey information and transmit the challenge information to the client according to the embodiments. Further, the processor may receive action information (or performance information) of the user with respect to the challenge information. The challenge information may represent a challenge including at least one of taking medicine, exercising, or drinking water. Action information may indicate whether a challenge is performed.

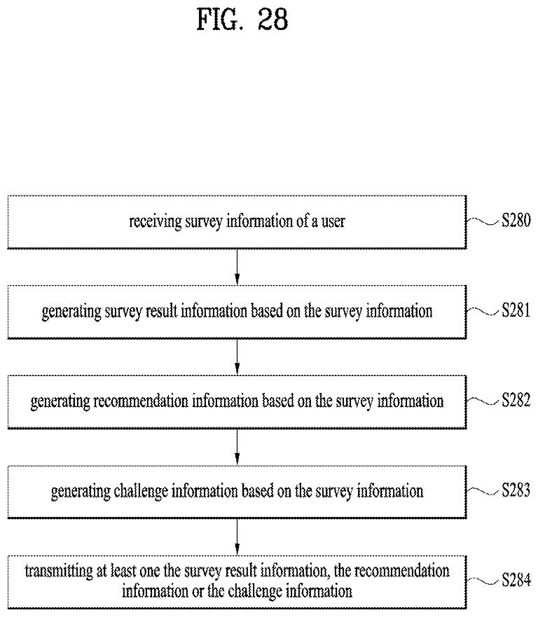

FIG. 28 illustrates a method of providing the online platform service according to embodiments.

The method of providing the online platform service according to the embodiments may be performed by the client, the server, or the database shown in FIGS. 25, 26A and 26B. Regarding the method according to the embodiments, the UI screens described with reference to FIGS. 2 to 21 may be displayed on a user terminal (or the client), and data input by the user may be transmitted and received between the client, the server and the database.

Referring to FIG. 28, the method of providing the online platform service according to the embodiments may include a step (S280) of receiving survey information from a user, a step (S281) of generating survey result information based on the user survey information, a step (S282) of generating recommendation information based on the user survey information, a step (S283) of generating challenge information based on the user survey information, and/or a step (S284) of transmitting at least one of the survey result information, the recommendation information, or the challenge information. Any one or more of the steps may be omitted, and the order of the steps may be changed.

Step S280 is a step of receiving survey information input by a user through the screens described with reference to FIGS. 6, 7, and 8. User survey information may be input from the client according to the embodiments and transmitted to the server or the database. The server or the database may receive the user survey information, store/manage the same, and generate data based on the survey information. The user survey information may include information indicating the age of the user, information indicating nutritional components consumed by the user, and information on the sex of the user, and may further include information shown in FIGS. 6 to 8.

Step S281 is a step of generating survey result information displayed on the screen described with reference to FIG. 9. The server or the database may generate the survey result information based on the user survey information. The survey result information may include information on cautions on intake of nutritional components and information on insufficient nutritional components, and may further include other information shown in FIG. 9.

Step S282 is a step of generating recommendation information displayed on the screens described with reference to FIGS. 4 and 17. The server or the database may generate the recommendation information based on client survey information. The recommendation information may be displayed on the screens as shown in FIGS. 4 and 17. The recommendation information may include information on product combinations. The information on product combinations represents information on products that are good to consume together. The recommendation information may further include keyword information related to symptoms that can be improved by products. The recommendation information may further include other information shown in FIGS. 4 and 17.

Step S283 is a step of generating challenge information displayed on the screens described with reference to FIGS. 3 and 20. The server or the database may generate the challenge information based on the client survey information. The challenge information may be displayed on the screens as shown in FIGS. 3 and 20. The challenge information may include at least one of taking medicine, exercising, or drinking water. The challenge information may further include other information shown in FIGS. 4 and 17.

Step S284 is a step of transmitting at least one of the survey result information, the recommendation information, or the challenge information. The apparatus (the client, the server, or the database) according to the embodiments may transmit information generated in the foregoing steps. For example, the server or the database may transmit information to the client. The client may display the received information on a screen by an application program installed therein. As shown in FIGS. 6 to 8, 9, 4, 17, 3 and 20, the received information may be displayed on the screen of the client.

Accordingly, the online prescription platform method and apparatus according to the embodiments have an effect of providing reliable health information accurately and efficiently based on AI services based on data accumulated from users and experts. The online prescription platform method and apparatus according to the embodiments have an effect of providing health and nutrition information optimized for personal information of users by overcoming limitations of using unverified information in individual markets. The online prescription platform method and apparatus according to the embodiments can process health information desired by a user using accurate professional data based on information received from users and experts. The online prescription platform method and apparatus according to the embodiments can provide users with accurate prescription and diagnosis information by training data accumulated in the platform using an AI algorithm. The online prescription platform method and apparatus according to the embodiments have an effect of providing an online platform service by overcoming limitations of offline markets. The online prescription platform method and apparatus according to the embodiments have an effect of implementing a UI/UX that improves user convenience.

The embodiments have been described in terms of a method and/or an apparatus, and description of the method and description of the apparatus may be applied interchangeably.

For convenience of description, drawings have been separately described, but it is also possible to implement a new embodiment by merging the embodiments described in respective figures. In addition, designing a computer-readable recording medium in which programs for executing the previously described embodiments are recorded according to the need of those skilled in the art falls within the scope of the embodiments. As described above, the apparatus and method according to the embodiments are not limited to the configuration and method of the embodiments described above, but all or some the embodiments may be selectively combined such that various modifications can be made. Although preferred embodiments have been shown and described, embodiments are not limited to the specific embodiments described above, and various modifications are possible by those skilled in the art to which the present invention pertains without departing from the gist of the embodiments claimed in the claims. These modifications should not be individually understood from the technical spirit or prospects of the embodiments.

Various components of the apparatus of the embodiments may be implemented by hardware, software, firmware or a combination thereof. Various components of the embodiments may be implemented as one chip, for example, as one hardware circuit. According to embodiments, components according to the embodiments may be implemented as separate chips. Depending on the embodiments, at least one of the components of the apparatus according to the embodiments may be composed of one or more processors capable of executing one or more programs, and the one or more programs may execute any one or more of operations/methods according to the embodiments or may include instructions for performing the operations/methods. Executable instructions for performing methods/operations of the apparatus according to the embodiments may be stored in a non-transitory CRM or other computer program products configured for execution by one or more processors, or may be stored in a transitory CRM or other computer program products configured for execution by one or more processors. In addition, the memory according to the embodiments may be used as a concept including not only a volatile memory (e.g., RAM) but also a non-volatile memory, a flash memory, a PROM, and the like. Further, those implemented in the form of a carrier wave such as transmission through the Internet may be included. In addition, a processor-readable recording medium is distributed to computer systems connected through a network such that processor-readable code can be stored and executed in a distributed manner.

In this specification, “I” and “,” should be interpreted as “and/or”. For example, “A/B” may be interpreted as “A and/or B” and “A, B” may be interpreted as “A and/or B”. Additionally, “A/B/C” may mean “at least one of A, B and/or C”. Further, “A, B, C” may mean “at least one of A, B and/or C”. Additionally, “or” should be interpreted as “and/or” in this specification. For example, “A or B” may include 1) “only A”, 2) “only B”, and/or 3) “both A and B”. In other words, “or” should be interpreted as “additionally or alternatively” in the specification.

Terms such as “first” and “second” may be used to distinguish various components of the embodiments. However, interpretation of various components according to the embodiments should not be limited by the above terms. These terms are merely used to distinguish one component from another. For example, a first user input signal may be referred to as a second user input signal. Similarly, the second user input signal may be referred to as the first user input signal. Use of these terms should be construed as not departing from the scope of the various embodiments. Although both the first user input signal and the second user input signal are user input signals, they do not mean the same user input signals unless the context clearly indicates otherwise.

Terms used to describe the embodiments are used for the purpose of describing specific embodiments and are not intended to limit the embodiments. As used in the description of the embodiments and in the claims, the singular expression is intended to include the plural expression unless the context clearly indicates otherwise. The expression “and/or” is used in a sense that includes all possible combinations between terms. The expression “includes” describes that there are features, numbers, steps, elements, and/or components, and does not imply that additional features, numbers, steps, elements, and/or components are not included. Conditional expressions such as “if ˜,” “when ˜,” etc., used to describe the embodiments are not limited to optional cases. When a specific condition is satisfied, a related operation is intended to be performed in response to the specific condition, or a related definition is intended to be interpreted.

Further, operations according to embodiments described in this specification may be performed by a transceiver including a memory and/or a processor according to embodiments. The memory may store programs for processing/controlling operations according to embodiments, and the processor may control various operations described in this specification. The processor may be referred to as a controller or the like. Operations in embodiments may be performed by firmware, software, and/or a combination thereof, and the firmware, software, and/or combination thereof may be stored in the processor or stored in the memory.

Meanwhile, the operations according to the above-described embodiments may be performed by a transmission device and/or a reception device according to the embodiments. The transmission/reception device may include a transmitter/receiver for transmitting/receiving media data, a memory for storing instructions (program code, algorithms, flowcharts and/or data) for processes according to embodiments, and a processor for controlling operations of the transmission/reception device.

The processor may be referred to as a controller or the like and may correspond to, for example, hardware, software, and/or a combination thereof. Operations according to the above-described embodiments may be performed by the processor. Further, the processor may be implemented as an encoder/decoder for the operations of the above-described embodiments.

What is claimed is:

1. An apparatus for providing an online platform service, the apparatus comprising:
a memory configured to store at least one instruction; and
a processor connected to the memory and configured to execute the at least one instruction to:
receive survey information of a user;
generate result information based on the survey information; and
transmit the result information.

2. The apparatus of claim 1, wherein the survey information includes information representing the age of the user, information representing nutritional supplements consumed by the user, and information representing the sex of the user.

3. The apparatus of claim 2, wherein the result information includes information representing cautions for intaking the nutritional supplements and information representing lack of nutritional supplements.

4. The apparatus of claim 3, wherein the processor is further configured to execute the at least instruction to:
generate recommendation information based on the survey information; and
transmit the recommendation information.

5. The apparatus of claim 4, wherein the recommendation information includes information representing a combination of products.

6. The apparatus of claim 5, wherein the recommendation information includes information representing keywords related to symptoms to be improved by the products.

7. The apparatus of claim 6, wherein the processor is further configured to execute the at least instruction to:
generate challenge information based on the survey information;
transmit the challenge information; and
receive action information of the user for the challenge information.

8. The apparatus of claim 7, wherein the challenge information represents a challenge including at least one of taking medicines, exercising or drinking water, and
wherein the action information represents whether the challenge is carried out.

9. A method of providing an online platform service, the method comprising:
receiving survey information of a user from a client;
generating result information based on the survey information; and
transmitting the result information to the client.

10. The method of the claim 9, wherein the survey information includes information representing the age of the user, information representing nutritional supplements consumed by the user and information representing the sex of the user.

11. The method of the claim 10, the method further comprising:
generating recommendation information based on the survey information; and
transmitting the recommendation information.

12. The method of the claim 11, the method further comprising:
generating challenge information based on the survey information;
transmitting the challenge information; and
receiving action information of the user for the challenge information.
